(19) United States
(12) Patent Application Publication
(10) Pub. No.: US 2024/0123249 A1
(43) Pub. Date: Apr. 18, 2024

(54) ANALGESIA VIA LOW AND ULTRA-LOW FREQUENCY MAGNETIC OR ELECTROMAGNETIC FIELDS AND RELATED SYSTEMS AND METHODS

(71) Applicant: EMulate Therapeutics, Inc., Bellevue, WA (US)

(72) Inventors: Bennett Michael Butters, Lacey, WA (US); Xavier A. Figueroa, Seattle, WA (US)

(21) Appl. No.: 18/487,017

(22) Filed: Oct. 13, 2023

Related U.S. Application Data
(60) Provisional application No. 63/416,384, filed on Oct. 14, 2022.

(51) Int. Cl. A61N 2/00; A61N 2/02

(52) U.S. Cl. CPC A61N 2/008; A61N 2/02

(57) ABSTRACT

Disclosed herein are methods and systems for reducing pain and/or providing an analgesic effect, using the administration of ultra-low radio frequency energy (ulRFE®). In some embodiments, ulRFE® is administered using the EMulate Voyager system. In some embodiments, the system comprises a battery-operated controller, an electromagnetic coil, and a battery charger. In some of these embodiments the system utilizes a single signal. In other embodiments, the system utilizes two or more signals, each signal derived from a different molecule. In some of the other embodiments, one or more of the signals are derived from the same molecule. In some embodiments, the system utilizes three or more signals derived from three or more different molecules, e.g., three signals, four signals, five signals, or more.

### CROSS-REFERENCE TO RELATED APPLICATIONS

This application claims priority to U.S. Provisional Patent Application No. 63/416,384, filed Oct. 14, 2022, which is incorporated herein in by reference in its entirety.

### BACKGROUND

Radio frequency energy (RFE) exposure in the 3 kHz to 3,000 GHz range has a measurable effect on human cells, and electromagnetic (EM) radiation in the RF range can impact cellular function in vitro and in vivo, without tissue heating. The magnetic field component of radiofrequency waves on living cells is likely a direct mechanism, as even weak magnetic fields affect cell function. The hypothesis that molecular interaction has a stronger EM component than previously thought is supported by computational evidence. In addition, molecules in solution generate a weak magnetic field as they stretch, twist, tumble and vibrate in an aqueous medium, and these magnetic fields are exceptionally weak, in the order of femto-Tesla (fT) in strength. These magnetic fields (as well as the electrostatic charge on the molecules) may be important for molecular recognition and non-covalent binding in many biological processes.

In the field of pain management, the use of opioids has become an essential tool, but the addictive and health-impacting effects of opioid misuse result in approximately 70,000 deaths per year in the US alone. Alternatives exist to opioids, but do not provide substantial pain relief. New methods and technologies to safely and effectively reduce pain sensation, either as a monotherapy or in combination with low dose and/or safe pain medications, are urgently needed in populations suffering from acute and chronic pain.

### SUMMARY

Provided herein in some embodiments are methods for reducing pain by administering ultra-low radio frequency energy (ulRFE®) to a subject with pain. In some embodiments, the pain is one or more of visceral pain (e.g., bowl and/or ureter obstructions, smooth-muscle cramping, angina, and pancreatitis), neuropathic pain (e.g., diabetic neuropathy, phantom limb pain, and post-herpetic neuralgia), and inflammatory pain (e.g., bursitis, arthritis, migraine, cluster headaches, and ankylosing spondylitis). In some embodiments, ulRFE® is administered using the EMulate Voyager system. In some embodiments, the system comprises a battery-operated controller, an electromagnetic coil, and a battery charger. In some of these embodiments the system utilizes a single signal. In other embodiments, the system utilizes two or more signals, each signal derived from a different molecule. In some of the other embodiments, one or more of the signals are derived from the same molecule. In some embodiments, the system utilizes three or more signals derived from three or more different molecules, e.g., three signals, four signals, five signals, or more.

Also provided herein in some embodiments are methods for providing an analgesic effect by administering ulRFE® to a subject with pain. In some embodiments, the pain is one or more of visceral pain (e.g., bowl and/or ureter obstructions, smooth-muscle cramping, angina, and pancreatitis), neuropathic pain (e.g., diabetic neuropathy, phantom limb pain, and post-herpetic neuralgia), and inflammatory pain (e.g., bursitis, arthritis, migraine, cluster headaches, and ankylosing spondylitis). In some of these embodiments, ulRFE® is administered using the EMulate Voyager system. In some embodiments, the system comprises a battery-operated controller, an electromagnetic coil, and a battery charger. In some of these embodiments the system utilizes a single signal. In other embodiments, the system utilizes two or more signals, each signal derived from a different molecule. In some of the other embodiments, one or more of the signals are derived from the same molecule. In some embodiments, the system utilizes three or more signals derived from three or more different molecules, e.g., three signals, four signals, five signals, or more.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present technology. Accordingly, various elements may be arbitrarily enlarged to improve legibility. For ease of reference, throughout this disclosure identical reference numbers may be used to identify identical, or at least generally similar or analogous components or features.

FIG. 1 is a diagram of a system in use on a canine patient;

FIG. 2 is another diagram of the system of FIG. 1;

FIG. 3 is a diagram of variations of coils used for providing electromagnetic or magnetic field treatment;

FIG. 4 is a diagram of variations of shapes and sizes of coils used for providing electromagnetic or magnetic field treatment;

FIGS. 5A-5B are views of the manufacture of a cable for the system;

FIG. 6 is a view of a connector for the cable;

FIG. 7 is a schematic view of the connector for the cable;

FIG. 8 is a flow diagram of a method of manufacturing a coil for the system;

FIG. 9 is an exploded view of a housing of a controller for the system;

FIGS. 10A-10E are electrical schematics of microprocessor circuitry for the controller;

FIG. 11 is an electrical schematic of memory for the controller;

FIG. 12 is an electrical schematic of various components for the controller;

FIG. 13 is an electrical schematic of an LCD interface for the controller;

FIGS. 14A-14C are electrical schematics of cognate generator circuitry for the controller;

FIGS. 15A-15B are electrical schematics of power regulation circuitry for the controller;

FIG. 16 is flow diagram of a method of operating the system;

FIGS. 17A-17B show diagrams of an example apparatus for securing the therapy system to the cranium of a human patient;

FIG. 18 is a diagram of a rat cage exposed to a system via a side view of the cage;

FIG. 19 is a representative graph showing the results of an oxaliplatin induced neuropathic allodynia pain model;

FIGS. 20A-20B are representative graphs showing the results of a carrageenan induced mechanical hyperalgesia pain model;

FIG. 21 is a representative graph showing the results of a trinitrobenzene sulfonic acid (TNBS) colonic sensitivity model of visceral pain;

FIGS. 22A-22B are representative graphs showing surgically induced tactile allodynia assessed using electronic von Frey; and

FIG. 23 is a representative graph showing the results of an acetic acid induced abdominal contraction model.

### DETAILED DESCRIPTION

The methods, apparatuses, devices, and systems described herein illustrate several embodiments of an ultra-low radio frequency energy (ulRFE®) technology-based delivery mechanism to provide pain relief and/or an analgesic effect in a subject having pain.

As set forth in the examples herein, a ulRFE® signal generated using the EMulate Voyager system was administered to a group of subjects with pain. During the study, multiple subjects exhibited positive responses to the treatment with no significant toxicity.

Based on these results, provided herein in some embodiments are methods of reducing pain and/or providing an analgesic effect in a subject in need thereof comprising administering to the subject a ulRFE® signal. Also provided herein is the use of a system capable of generating a ulRFE® signal to administer ulRFE® to a subject with pain. In some embodiments, the system uses a signal derived from a single molecule. In some embodiments, the system uses two signals derived from two different molecules. In some embodiments, the system uses three or more signals derived from three or more different molecules. Devices, systems, apparatuses, and kits for carrying out the disclosed methods and uses are also provided.

The terms below generally have the following definitions unless indicated otherwise. Such definitions, although brief, will help those skilled in the relevant art to more fully appreciate aspects of the present technology based on the detailed description provided herein. Other definitions are provided above. Such definitions are further defined by the description of the present technology as a whole (including the claims) and not simply by such definitions.

“Ultra-low radio frequency energy” or “ulRFE®” refers to magnetic fields having frequencies in the range of approximately 1 Hz (or less) to 22 kHz.

“Cognate” refers to a ulRFE® containing a record of the electromagnetic properties of a molecule, including, without limitation, molecules that are therapeutic compounds, such as, siRNA, nucleic acids, proteins, or chemicals.

“Magnetic shielding” refers to shielding that decreases, inhibits or prevents passage of magnetic flux as a result of the magnetic permeability of the shielding material.

“Electromagnetic shielding” refers to, e.g., standard Faraday electromagnetic shielding, or other methods to reduce passage of electromagnetic radiation.

“Faraday cage” refers to an electromagnetic shielding configuration that provides an electrical path to ground for unwanted electromagnetic radiation, thereby quieting an electromagnetic environment.

“Time-domain signal” or “time-series signal” refers to a signal with transient signal properties that change over time.

“Sample-source radiation” refers to magnetic flux or electromagnetic flux emissions resulting from molecular motion of a sample, such as the motions of larger molecular groupings like proteins, and the effects these motions have on surface charge. Because sample source radiation may be produced in the presence of an injected magnetic-field stimulus, it may also be referred to as “sample source radiation superimposed on injected magnetic field stimulus.”

“Stimulus magnetic field” or “magnetic-field stimulus” refers to a magnetic field produced by injecting (applying) to magnetic coils surrounding a sample, one of a number of electromagnetic signals that may include (i) white noise, injected at voltage level calculated to produce a selected magnetic field at the sample of between 0 and 1 G (Gauss), (ii) a DC offset, injected at voltage level calculated to produce a selected magnetic field at the sample of between 0 and 1 G, and/or (iii) sweeps over a low-frequency range, injected successively over a sweep range between at least about 0-1 kHz, and at an injected voltage calculated to produce a selected magnetic field at the sample of between 0 and 1 G. The magnetic field produced at the sample may be readily calculated using known electromagnetic relationships, knowing a shape and number of windings in an injection coil, a voltage applied to coils, and a distance between the injection coils and the sample.

A “selected stimulus magnetic-field condition” refers to a selected voltage applied to a white noise or DC offset signal, or a selected sweep range, sweep frequency and voltage of an applied sweep stimulus magnetic field.

“White noise” refers to random noise or a signal having simultaneous multiple frequencies, e.g., white random noise or deterministic noise. Several variations of white noise and other noise may be utilized. For example, “Gaussian white noise” is white noise having a Gaussian power distribution. “Stationary Gaussian white noise” is random Gaussian white noise that has no predictable future components. “Structured noise” is white noise that may contain a logarithmic characteristic which shifts energy from one region of the spectrum to another, or it may be designed to provide a random time element while the amplitude remains constant. These two represent pink and uniform noise, as compared to truly random noise which has no predictable future component. “Uniform noise” means white noise having a rectangular distribution rather than a Gaussian distribution.

“Frequency-domain spectrum” refers to a Fourier frequency plot of a time-domain signal.

“Spectral components” refers to singular or repeating qualities within a time-domain signal that can be measured in the frequency, amplitude, and/or phase domains. Spectral components will typically refer to signals present in the frequency domain.

A “subject” as used herein is an animal, preferably a mammal. In some embodiments, the subject is a human.

A “subject in need thereof” as used herein refers to a subject who has been experiencing pain. In some embodiments, the pain is one or more of visceral pain, neuropathic pain, and inflammatory pain.

In those embodiments of the methods and uses provided herein wherein a conventional pain medication is administered in combination with administration of the ulRFE® signal, any pain medication approved for the type of pain being treated can be used.

In some embodiments of the methods and uses provided herein, ulRFE® is administered using the EMulate Voyager system. As used herein, and described in more detail below, the terms “magnetic field,” “electromagnetic field” and similar terms are used interchangeably to describe the presentation of ulRFE® to a selected region to produce biological effects, where the presented ulRFE® has a characteristic reflecting that of a specific drug, chemical or other agent.

In some of these embodiments, the system is used to administer a ulRFE® signal obtained from a single molecule, or from one or more different molecules. The cognate sample for the ulRFE® signal can be obtained from one or more pharmacological compound. The one or more pharmacological compound may be an opioid analgesic, a mixed opioid agonist/antagonist, a COX-2 inhibitor, a muscle relaxant, a steroid (e.g., a corticosteroid), an anxiolytic, an antidepressant, an anticonvulsant, and/or a nonsteroidal anti-inflammatory drug (NSAID). Non-limiting examples of pharmacological compounds that may be used to obtain the ulRFE® signal include opioid analgesics such as, e.g., morphine, (−)U50,488H, oxycodone, oxycodone HCl, hydrocodone, oxycontin, fentanyl, propoxyphene, pentazocine, tapentadol, oxymorphone hydrochloride, hydromorphone, terephthalate oxycodone HCl, meperidine, meperidine HCl, tramadol, codeine, buprenorphine, methadone, and morphine sulfate; mixed opioid agonists/antagonists such as, e.g., cannabidiol, pentazocine/naloxone, butorphanol, and nalbuphine; COX-2 inhibitors such as, e.g., celecoxib; muscle relaxants such as, e.g., baclofen, tizanidine, carisoprodol, methocarbamol, orphenadrine, dantrolene, chlorzoxazone, and cyclobenzaprine; corticosteroids such as, e.g., cortisone, hydrocortisone, prednisone, dexamethasone, betamethasone, prednisolone, methylprednisolone, and triamcinolone; anxiolytics such as, e.g., diazepam, triazolam, alprazolam, and lorazepam; antidepressants such as, e.g., duloxetine, milnacipran, desipramine, bupropion, imipramine, amitriptyline; anticonvulsants such as, e.g., carbamazepine, tiagabine, pregabalin, lamotrigine, topiramate, clonazepam, and gabapentin; and/or NSAIDs such as, e.g., ibuprofen, nabumetone, fenoprofen, piroxicam, tolmetin, ketorolac, sulindac, diflunisal, mefenamic acid, etodolac, meloxicam, flurbiprofen, naproxen, aspirin, acetaminophen, and indomethacin. In some embodiments, the one or more pharmacological compound is duloxetine, morphine, (−)U50,488H, and/or indomethacin. The terms “ulRFE®”, “cognate” and “signal” are at times used interchangeably herein. Every molecule has a specific and unique electrostatic surface potential. Electrostatic surface potential is a critically important property of a molecule; it is a key factor in how a molecule interacts with (and in) a biological system. The electrostatic surface potential of a molecule can be measured and recorded to derive a cognate using “Super Conducting Quantum Interference Device” (SQUID)-based technology. Transducing these highly precise ulRFE® profiles (cognates) into biological systems can produce precise biological responses. Transduction of these cognates induces selective charge forcings in a defined bioactive target, thus altering cell dynamics, which can produce biological effects.

The EMulate Voyager system can produce low-level radio frequency energy (RFE) that induces a biologic response to pain. The encrypted RFE signal is embedded in the firmware of the Voyager controller of the system during manufacturing. For example, using RFE derived from a muscle relaxant, the Voyager therapy may relieve muscle tension and/or muscle spasms.

In some embodiments, ulRFE® is administered using the EMulate Voyager system, and in some of these embodiments, the system utilizes a single signal derived from a single molecule. In other embodiments, the system utilizes a first signal and a second signal, each signal derived from a different molecule. In some of the other embodiments, the first and second signals are derived from the same molecule. In some embodiments, the first and second signal the first and second signals are administered over approximately the same time course, i.e., the first and last administrations of each occur around the same time. In other embodiments, the first signal may be administered to the subject before the second signal. For example, a subject may have received the first signal for some period prior to administration of the second signal, or vice versa. Similarly, administration of one signal may continue after administration of the other has ceased. For example, administration of the first signal may continue after the last administration of the second signal, or vice versa.

In some embodiments, ulRFE® is administered using the EMulate Voyager system, and in some of these embodiments, the system utilizes three or more signals, each derived from a different molecule. In other embodiments, the system utilizes three or more signals, each derived from the same molecule. In some embodiments, the three or more signals are administered over approximately the same time course, i.e., the first and last administrations of each occur around the same time. In other embodiments, administration of the three or more signals occurs subsequently or alternatingly. For example, a subject may receive a first signal for a period of time, then a second signal for a period of time after the administration of the first signal has ceased, and subsequently a third signal, after administration of the second signal has ceased. In some embodiments, when the system utilizes three or more signals, a subject may receive two signals concurrently, e.g., over approximately the same course, and one signal subsequently, e.g., over a different, subsequent course. For example, a subject may receive a first signal and a second signal over approximately the same course, and, after administration of the first and second signals has ceased, the subject may receive a third signal. Similarly, administration of one signal may continue after administration of the other two has ceased. For example, administration of the first signal may continue after the last administration of the second and third signals.

In some embodiments of the methods and uses provided herein, the ulRFE® signal is administered alone or in combination with one or more conventional pain medications, e.g., opioid analgesics, mixed opioid agonists/antagonists, muscle relaxants, corticosteroids, COX-2 inhibitors, anxiolytics, antidepressants, or nonsteroidal anti-inflammatory drugs (NSAIDs). For example, a ulRFE® signal may be administered in combination with opioid analgesics including, but not limited to, morphine, (−)U50,488H, oxycodone, oxycodone HCl, hydrocodone, oxycontin, fentanyl, propoxyphene, pentazocine, tapentadol, oxymorphone hydrochloride, hydromorphone, terephthalate oxycodone HCl, meperidine, meperidine HCl, tramadol, codeine, buprenorphine, methadone, and morphine sulfate; mixed opioid agonists/antagonists including, but not limited to, cannabidiol, pentazocine/naloxone, butorphanol, and nalbuphine; COX-2 inhibitors including, but not limited to, celecoxib; muscle relaxants including, but not limited to, baclofen, tizanidine, carisoprodol, methocarbamol, orphenadrine, dantrolene, chlorzoxazone, and cyclobenzaprine; corticosteroids including, but not limited to, cortisone, hydrocortisone, prednisone, dexamethasone, betamethasone, prednisolone, methylprednisolone, and triamcinolone; anxiolytics including, but not limited to, diazepam, triazolam, alprazolam, and lorazepam; antidepressants including, but not limited to, duloxetine, milnacipran, desipramine, bupropion, imipramine, amitriptyline; anticonvulsants including, but not limited to, carbamazepine, tiagabine, pregabalin, lamotrigine, topiramate, clonazepam, and gabapentin; and/or NSAIDs including, but not limited to, ibuprofen, nabumetone, fenoprofen, piroxicam, tolmetin, ketorolac, sulindac, diflunisal, mefenamic acid, etodolac, meloxicam, flurbiprofen, naproxen, aspirin, acetaminophen, and indomethacin.

In some embodiments, when the ulRFE® signal is administered in combination with one or more conventional pain medications, e.g., opioid analgesics, mixed opioid agonists/antagonists, muscle relaxants, corticosteroids, COX-2 inhibitors, anxiolytics, antidepressants, or nonsteroidal anti-inflammatory drugs (NSAIDs), the ulRFE® signal and the one or more pain medications are administered over approximately the same time course, i.e., the first and last administrations of each occur around the same time. In other embodiments, one may be administered to the subject before the other. For example, a subject receiving conventional pain medication may have received the pain medication for some period prior to the first ulRFE® administration, or vice versa. Similarly, administration of one may continue after the other has ceased. For example, ulRFE® administration may continue after the last administration of conventional pain medication, or vice versa.

In some embodiments of the methods and uses provided herein, ulRFE® is administered continuously during the treatment period, i.e., 24 hours/day (except for brief periods for medical procedures and personal hygiene). In other embodiments, ulRFE® is administered non-continuously, e.g., at specific intervals or at specific intervals throughout the conventional pain medication treatment period. In some embodiments, ulRFE® is administered in multiple cycles of the same or different lengths, e.g., multiple cycles of 4 weeks each.

Provided herein in some embodiments are methods of reducing pain and/or providing an analgesic effect in a subject in need thereof comprising administering to the subject a ulRFE® signal using the EMulate Voyager system. In some embodiments, the methods of reducing pain and/or providing an analgesic effect in a subject in need thereof further comprises administering a ulRFE® signal using the EMulate Voyager system and one or more conventional pain medications.

Use of ulRFE® may avoid problems of drug-based delivery, such as the ability of drugs to reach their intended target(s). For example, magnetic fields in the radio frequency range (derived from an alternating current (AC) source between 3 kHz to 3000 GHz) of low power and low frequency sufficiently penetrate tissue(s), ensuring access to areas that are poorly perfused. As such, ulRFE® technologies employ signals in a frequency range of 0-22 kHz, which may modulate specific regulatory, metabolic or other pathways in humans, animals, and plants by directly regulating the production of protein, starch, sugar, fat, and other molecules in cells, or altering other cellular functions such as cell division.

EMulate Voyager ulRFE® technology may be implemented by medical professionals and/or researchers to identify effective, safe, and less expensive alternatives to pain medications by developing ulRFE® transduction mechanisms for some applications. Applicant has disclosed, in related patents and patent applications noted herein, systems and methods for detecting and recording molecular cognates from chemical, biochemical, or biological molecules or from chemical, biochemical, or biological agents. In some implementations, the recordings represent molecular cognates of the chemical, biochemical, or biological molecules or agents used to provide treatment for pain, ailments or other adverse health conditions. The methods and systems disclosed herein may be configured to deliver the effect of chemical, biochemical, or biologic treatment to a human and/or animal, without the use of drugs or chemicals, by delivering cognates derived from particular chemicals, biochemical, or biologics or their respective effects. Thus, the methods and systems allow humans and/or animals to receive an electronic exposure to electromagnetic or radio frequency energy with, for example, the click of a button. The embodiments of the systems and methods describe a system that is non-invasive, non-thermal, non-ionizing and mobile.

In some embodiments, the Voyager system comprises three components: a battery-operated controller, an electromagnetic coil, and a battery charger. In some embodiments, the electromagnetic coil is worn on the subject's head and is connected to the battery-operated controller. In some embodiments, the coil can come in a variety of sizes so that it can fit any subject's head. In other of these embodiments, a cap or headband may be worn over the coil to hide if from view or to hold it in place as needed or as desired. In some embodiments, the Voyager system does not require the subject to shave his or her head or any other special preparation for use. In these embodiments, wearing the electromagnetic coil on the head may be useful for treating pain associated with headache or migraine, for example.

In other embodiments, the electromagnetic is worn on the subject's extremity and is connected to the battery-operated controller. For example, the electromagnetic coil may be worn on the subject's arm, such as around their shoulder, elbow, or wrist. Likewise, the electromagnetic coil may be worn on the subject's leg, such as around their hip, knee, or ankle. The electromagnetic coil may also be worn around the subject's waist or neck. Suitable configuration of the electromagnetic coil include a wrap, a brace, a bracelet, and a collar, among others. In some embodiments, the coil can come in a variety of sizes so that it can fit any subject. The coil may be pre-sized to the subject to ensure proper fit. In some embodiments, the coil is sizable by the subject. For example, in the case of a wrap, the coil may be configured as a flexible flat coil that can be wrapped around the subject and secured by any means of attachment.

In other embodiments, the electromagnetic coil is a flat coil that may be placed on a surface other than the subject's head. Non-limiting examples of configurations of the electromagnetic coil include pads of various shapes sized to deliver the signal to the subject at a particular site of the subject's body experiencing pain such as a back pad, a neck pad, an arm pad or a leg pad. In some configurations, the pad is flexible so as to conform to a shape of at least a portion of the subject's body. In other configurations, the pad is stiff so as to provide support to at least a portion of the subject's body.

In some embodiments, the Voyager system provides easy and comfortable use. For example, the Voyager system can be used in a home or office environment, so that the subject can carry on with daily activities without disruption from use of the Voyager system. In some embodiments, each battery-operated controller has a battery-life of approximately 16 hours. In some of these embodiments, the subject is provided with two battery-operated controllers so that one unit may be charged using the battery charger (like a cell phone charger) while the other controller is in use. In some embodiments, recharging takes less than 2 hours, the battery-operated controller weighs only 2.7 ounces, and/or is approximately the size of a pager. In some of these embodiments, the battery-operated control is clipped to a belt or an arm band worn by the subject.

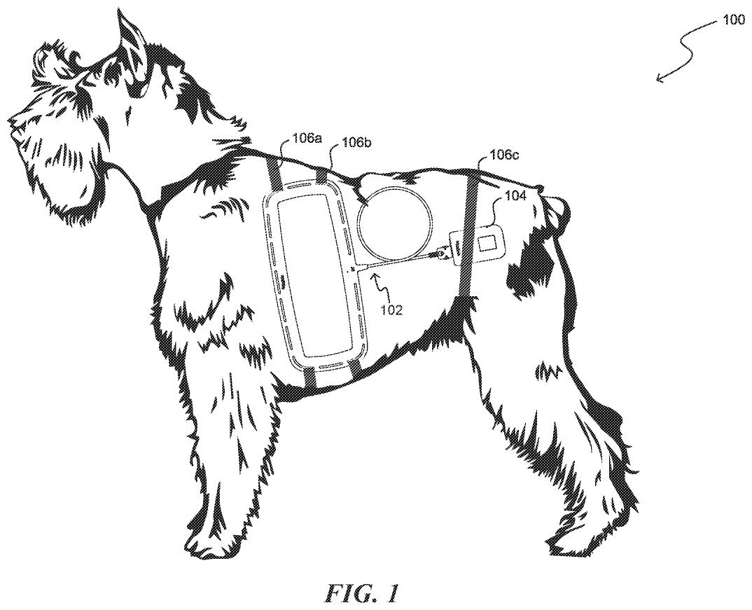

FIG. 1 illustrates an embodiment of a system 100 for applying ulRFE® cognates to an animal, such as a canine, to provide treatment, such as to selectively reduce pain and/or provide an analgesic effect. In some implementations, the system 100 may be used to treat pain by applying electromagnetic or magnetic fields to affected areas. These fields are induced or generated to expose an affected area to cognates derived from magnetic fields that emanate from drugs, chemicals or other agents. The acquisition of the cognates produced from drugs, chemicals or other agents is discussed in great detail in patent applications and patents that are co-owned by the assignees of the instant application. These patents and applications include U.S. Pat. Nos. 6,724,188, 6,995,558, 6,952,652, 7,081,747, 7,412,340, and 7,575,934; PCT Application Nos. PCT/US2009/002184, PCT/US2013/050165; and U.S. Patent Publication No. 2016/0030761A1, each of which is hereby incorporated by reference in its entirety.

The system 100 may provide various advantages over traditional treatments. For example, the system 100 may be portable and worn by humans or animals or kept near humans and/or animals as the situation requires.

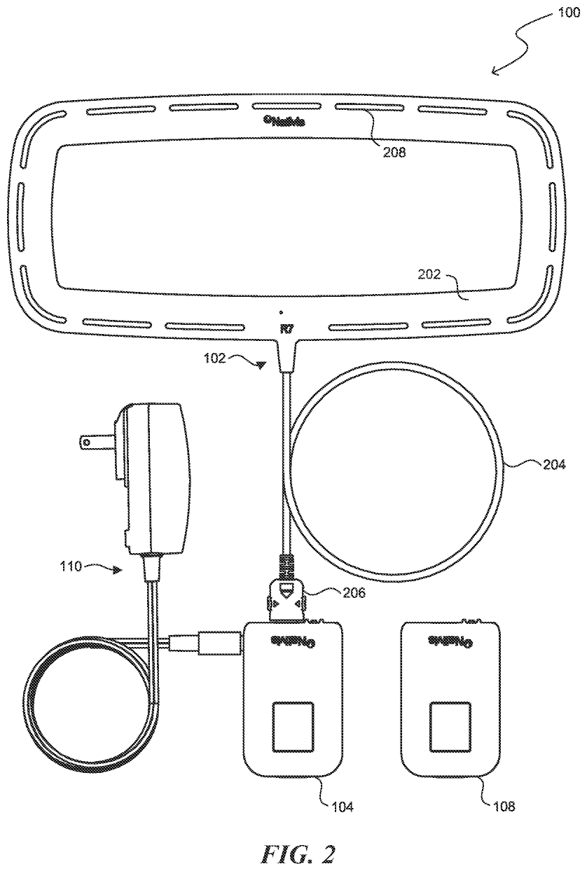

FIG. 2 illustrates the system 100 as it may be utilized. In addition to the transduction coil and cable 102 and controller 104 assemblies delivered to a user, the system 100 may also include additional coils, one or more additional controllers 108 and a battery charging device 110. For various security reasons, which are discussed below, each controller may be manufactured so that a housing for the controller cannot be opened easily.

The system 100 may also include a motion sensor (for example, accelerometer). The motion sensor can cause the controller 104 to issue an alert if sufficient undesirable motion is detected. (Of course, the motion sensor may be applied to any of the systems described above for similar purpose, such as when treatment recipient is sleeping, and moves sufficiently to cause a wearable coil 202 to potentially become dislodged, so that the alert or alarm can prompt the repositioning of the coil. The motion sensor and alarm can help ensure compliance with a treatment regime.)

In particular, the system 100 may include software stored in memory of the system (e.g., on-chip memory of a microprocessor, not shown). The software receives motion signal data from the motion sensor, which can reflect force vectors or measurements, over a period of time. The software then compares the force, direction and time of received motion data to stored rules or values to determine whether the received data represents an undesirable condition. If the system detects an undesirable condition, it can take remedial action, such as by issuing an alarm. If the system 100 includes wireless communication circuitry, the system can send an alert message to a remote monitoring facility. The system may also monitor and store global positioning information useful in determining the movement and position of livestock and field equipment.

The controller 104 can be formed of inexpensive components so as to reduce the overall cost of the system 100. Indeed, the system 100 can be configured to be disposable or of limited reusability. For example, the controller may have a system-on-chip (SoC) configuration whereby the SoC is a single semiconductor die that includes a microcontroller, memory, and analog amplifying circuitry, all monolithically formed. The controller 104 can include various types of power sources, such as a battery, capacitor, or even antenna(s) and associated circuitry so as to wirelessly obtain power that is then stored in a capacitor and used to drive the circuitry of the controller. Indeed, these and other power sources may be used for not only the system of FIG. 2, but all other systems and apparatus described herein.

### System Coil and Cable Assembly

In FIG. 2, the coil and cable assembly 102 includes an encapsulated coil 202, a cable 204, and a connector 206. The coil 202 includes one or more conductors configured to generate a magnetic or electromagnetic field from one or more cognates. As used herein, a drug or chemical-simulating cognate includes a cognate that approximately reproduces magnetic fields that emanate from one or more predetermined chemical, biochemical, and/or biological molecules or agents. The coil 202 may be configured to have various electrical characteristics. Additionally, the coil 202 may be enclosed in a plastic or other composite material to both protect the windings of the coil. As mentioned above, the system 100 may include more than one coil. Regardless of whether the system 100 is configured with one coil, or more than one coil, the coils can be flexible and malleable, can have a variety of shapes, can have different sizes or types, and can also include rigid coils. Advantageously, one or more of these coils can be externally secured to an animal to provide treatment, as opposed to subcutaneous insertion of the coil into an animal.

The controller 104 may be used in various environments. For example, the coil 202 may be placed in an animal stall, or on a bed, such as under a mattress pad of a veterinary or hospital bed or within the seat/seatback of a cart or wheelchair (or in a pillow), with the controller 104 removably attached to a frame of the cart, bed/wheelchair. As a result, a human or animal need only lie in the stall or bed to receive treatment, rather than have the coil 202 attached to the human or animal's body as described herein.

The controller 104 may store multiple cognates. The controller may then also include a software or hardware switch that allows a user to select one of the multiple cognates to be amplified and output by the controller, so that the controller may be used to generate an output of two or more cognates, such as with two matched coils (e.g., a Helmholtz coil pair), and may include two different channels, one for each of the two coils. The controller 104 can include phase control so as to control the two coils and ensure that they are in sync. Such phase control can take the form of a locking amplifier, phase lock loop circuitry, or other known means. As a result, the two coils can produce the same wireless ulRFE®, which can then be applied to a larger area.

Alternatively, the two coils can each include different geometries to account for application of the cognate to different portions of a target region, and/or to account for different geometries of the recipient. For example, if two different coils are to be positioned on a recipient's body, the coils can account for the geometries of the different locations on the body, and to account for different geometries of the target within the body (e.g., different top and side cross-sections of the same organ.)

Alternatively, rather than apply the same cognate to both coils, the controller 104 can store two or more different cognates, and apply one to coil and the other concurrently to another coil. Of course, the system 100 can include a selector that allows for both functions: applying the same cognate to both coils, or a different cognate to each of the two coils. Of course, the controller can apply two or more cognates serially, one after the other, and then loop back (e.g., apply cognates A, B and C serially in a sequence of A, B, C, A, B, . . . , though other sequences are possible). The time period for application of each cognate need not be the same, but could differ (e.g., cognate A applied for 15 minutes, B for 10 and C for 5, then the series repeats).

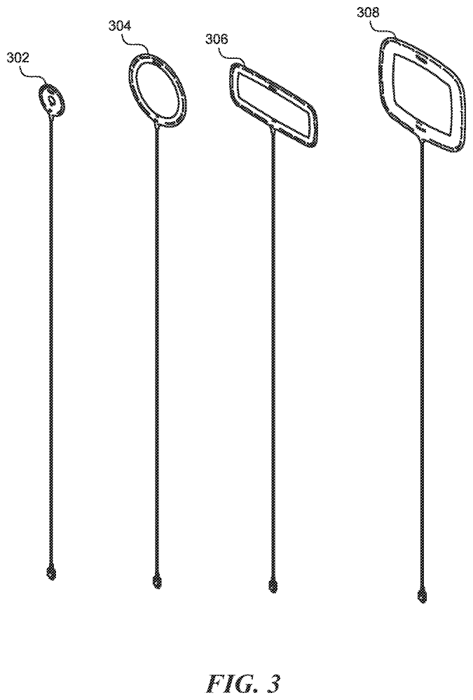

FIG. 3 illustrates diagrams of variations to the shape of the encapsulated coil 202. As illustrated, the coils used by the system 100 may include a small circular encapsulated coil 302, a large circular encapsulated coil 304, a rectangular encapsulated coil 306, a substantially square encapsulated coil 308, and/or another encapsulated coil sized and shaped to treat a particular part of the human's, mammal's, and/or animal's body. Each shape may provide advantages for treating particular parts of the body of the human, mammal, and/or animal.

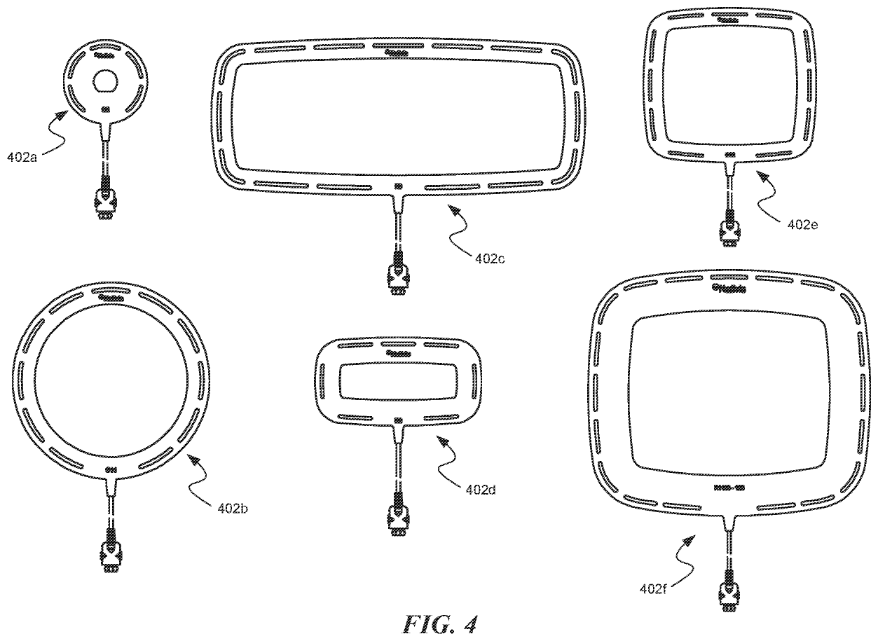

FIG. 4 illustrates examples of coils having various shapes and various dimensions. A variety of dimensions for the coils may be manufactured to more effectively apply treatment to areas that vary in size. Each of the coils 402a, 402b, 402c, 402d, 402e, and 402f can have inner and/or outer diameters or lengths, ranging from just a few centimeters to several feet, according to various implementations.

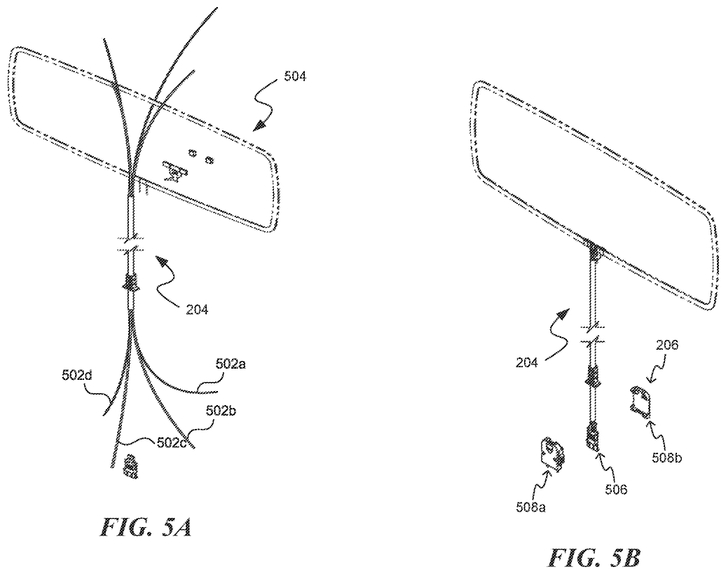

FIGS. 5A and 5B illustrate before and after diagrams of the cable 204 during manufacture. The cable 204 connects a coil, e.g., coil 202, to the connector 206 to enable the controller 104 to transmit various cognates to the coil. The cable 204 may include two or more conductors 502a, 502b, a shield 502c (also referred to as a “conductor shield”), and a strength-providing member 502d (collectively conductors 502). Each of the four conductors and members may be configured to perform a particular function. For example, conductors 502a and 502b may be electrically coupled to either end of the coil 504 to enable current to flow to and from the coil 504 to generate a magnetic field from the coil 504. Shield conductor 502c may be coupled to ground and be configured to provide electromagnetic shielding for the conductors 502a and 502b. Strength member 502d may be anchored to the coil 504 and to the connector 206 to provide strain relief to the conductors 502a-502c. In some implementations, the strength member 502d is manufactured with a shorter length than the other conductors so that the strength member 502d receives a majority of any strain applied between the coil 504 and the connector 206.

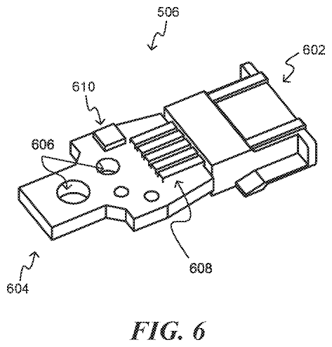

As illustrated in FIG. 5B, the connector 206 may include three parts, a connector core 506, and connector housings 508a and 508b. The connector housings 508a and 508b may encapsulate the connector core 506 to protect the traces and electronic devices carried by the connector core 506. FIG. 6 illustrates an implementation of the connector core 506. The connector core 506 has a controller end 602 and a cable end 604. The controller end 602 is configured to couple to the controller 104, and the cable end 604 is configured to provide an interface for the conductors 502. In some implementations, the strength member 502d may be anchored to one or more holes 606 to provide strain relief. The conductor core 506 may also carry a plurality of traces 608 to which the conductors 502a-c may be electrically coupled to facilitate communication with the controller 104.

As a security feature of the coil and cable assembly 102, the connector core 506 may also carry an integrated circuit 610. The integrated circuit 610 may be a microprocessor or may be a stand-alone memory device. The integrated circuit 610 may be configured to communicate with the controller 104 through the controller end 602 using communication protocols such as I2C, 1-Wire, and the like. The integrated circuit 610 may include a digital identification of the coil with which the connector core 506 is associated.

The digital identification stored on the integrated circuit 610 may identify electrical characteristics of the coil, such as impedance, inductance, capacitance, and the like. The integrated circuit 610 may also be configured to store and provide additional information such as the length of the conductor of the coil, physical dimensions of the coil, and number of turns of the coil. In some implementations, the integrated circuit 610 includes information to prevent theft or reuse in a knock-off system, such as a unique identifier, cryptographic data, encrypted information, etc. For example, the information on the integrated circuit 610 may include a cryptographic identifier that represents measurable characteristics of the coil and/or the identification of the integrated circuit. If the cryptographic identifier is merely copied and saved onto another integrated circuit, for example, by an unauthorized manufacturer of the coil and cable assembly, the controller 104 may recognize that the cryptographic identifier is illegitimate and may inhibit cognate transmissions. In some implementations, the integrated circuit stores one or more encryption keys, digital signatures, stenographic data or other information to enable communications and/or security features associated with public key infrastructure, digital copy protection schemes, etc.

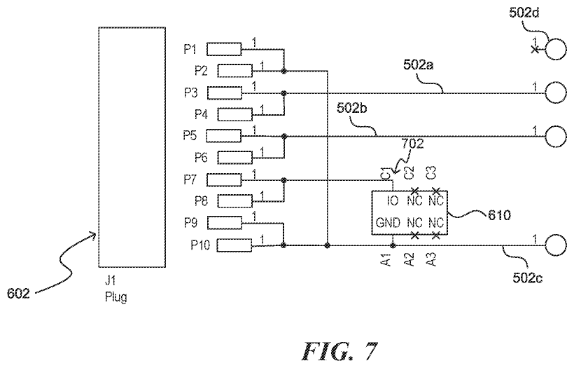

FIG. 7 illustrates a schematic diagram of the connector core 506. As shown, according to some implementations, the integrated circuit 610 may be configured to communicate with the controller 104 over a single wire, e.g., from input-output-pin 702.

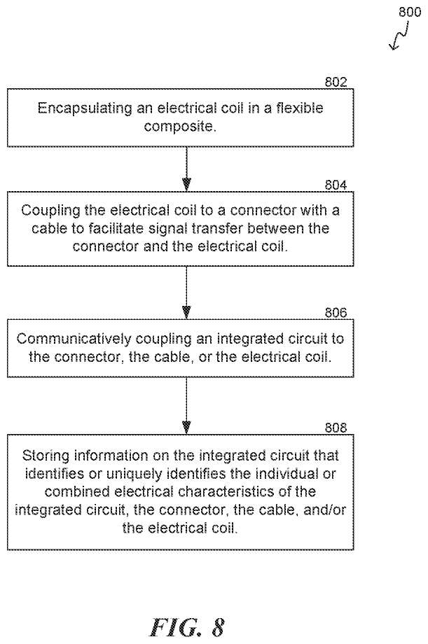

FIG. 8 illustrates a method 800 of manufacturing a coil and cable assembly, e.g., the coil and cable assembly 102, for use in providing a system that is non-invasive, non-thermal, non-ionizing and mobile.

At block 802, an electrical coil is encapsulated in a flexible composite. The flexible composite allows the electrical coil to be comfortably secured to, e.g., an animal to provide magnetic field treatment.

At block 804, the electric coil is coupled to a connector through a cable to facilitate secure transfer between the connector and the electrical coil. The cable may include multiple conductors that deliver signals between the connector and the electrical coil while providing mechanical strain relief to the signal carrying conductors.

At block 806, an integrated circuit is coupled to the connector, the cable, or the electrical coil. The integrated circuit may be coupled, for example, to the connector via one or more electrical conductors that may or may not also be coupled to the electrical coil.

At block 808, information is stored to the integrated circuit that identifies or uniquely identifies the individual or combined electrical characteristics of the integrated circuit, the connector, the cable, and/or the electrical coil. The information may be a hash or other cryptographically unique identifier that is based on information that can be unique to the integrated circuit and/or the remainder of the coil and cable assembly. This security feature can be used to prevent or deter unauthorized remanufacture of coil and cable assemblies that are compatible with the controller for the magnetic field delivery system. Additional security features are described herein, e.g., in connection with the operation of the controller for the system.

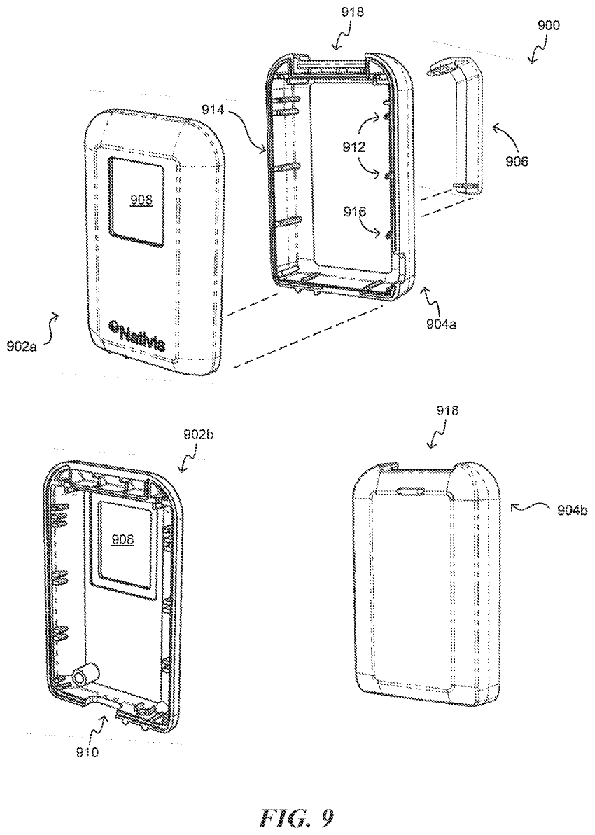

Referring briefly back to FIG. 2, the system 100 includes a controller 104 to provide an interface to the human and/or, to distribute and regulate drug and chemical-simulating cognates to the coil 202, and to prevent unauthorized copying and/or distribution of the drug or chemical-simulating cognates. According to various implementations, the controller 104 can include various features such as a housing, a processor, memory, visual and audio interfaces, in addition to other features which are described hereafter in FIGS. 9-15B.

FIG. 9 illustrates a housing 900 for the controller 104. The housing 900 may include three parts, a housing front 902 (inclusive of 902a, 902b), a housing back 904 (inclusive of 904a, 904b), and a clip 906. The housing front 902 may have a window 908 through which a visual interface may be viewed or manipulated. Although not shown, the housing front 902 may include various apertures through which buttons, dials, switches, light emitting indicators, and/or a speaker may pass or be disposed. The housing front 902 includes a cut-away or port 910 for coupling the controller 104 to the coil and cable assembly 102. The housing back 904 may include a number of pegs 912 for attaching/securing the housing back 904 to the housing front 902. While coupled together, the housing front 902 and the housing back 904 may form a seal along the edge 914, preventing water, moisture, dust, or other environmental elements from entering the housing 900. In some implementations, an adhesive or solvent is used to permanently bond the housing front 902 to the housing back 904 to deter or prevent unauthorized tampering with or viewing of the internal electronics, though in other implementations the front and back may be formed to permanently snap-fit together. As shown, the housing back 904 may include a cutout, aperture, or port 916 to allow connection to a recharging device or communication information to/from the controller 104. The clip 906 may be securely fastened or detachably coupled to slot 918 of the housing back 904 to secure or affix the controller 104.

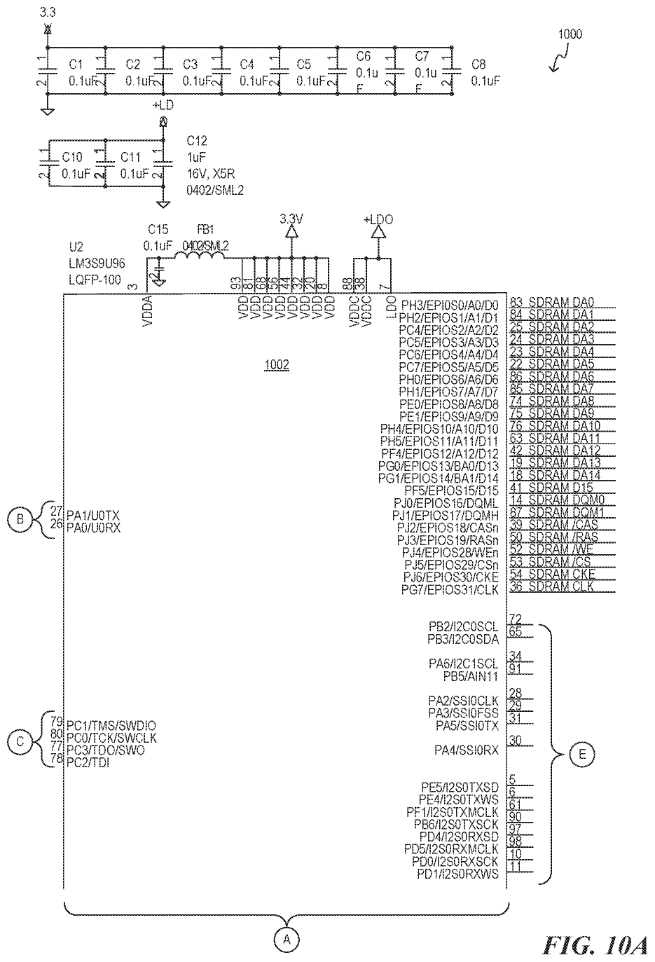

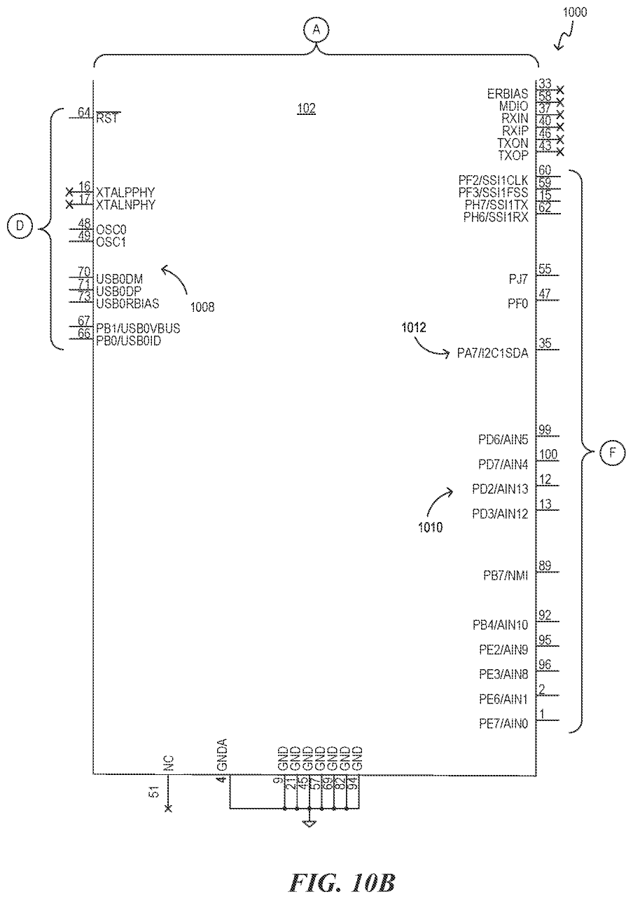

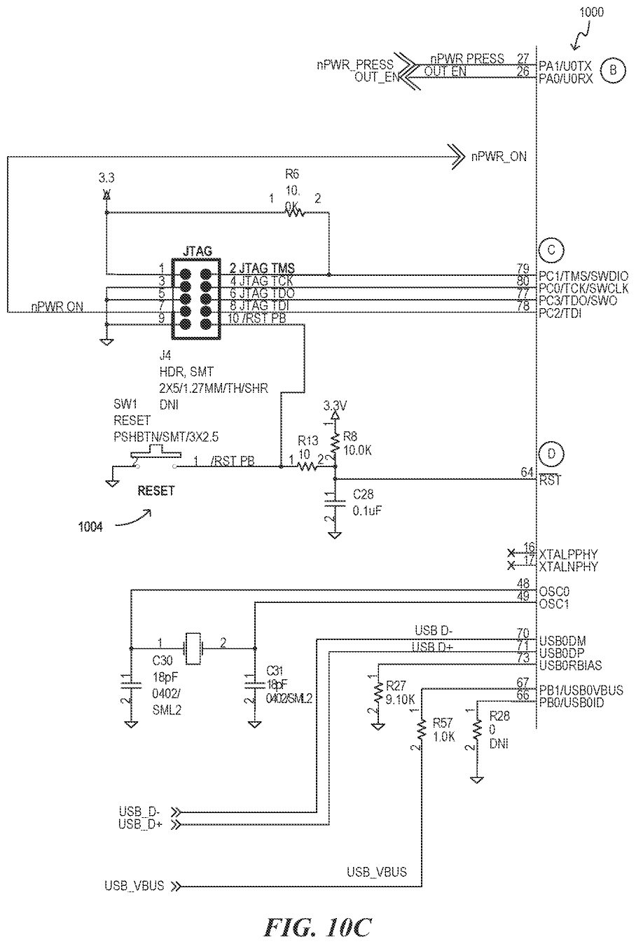

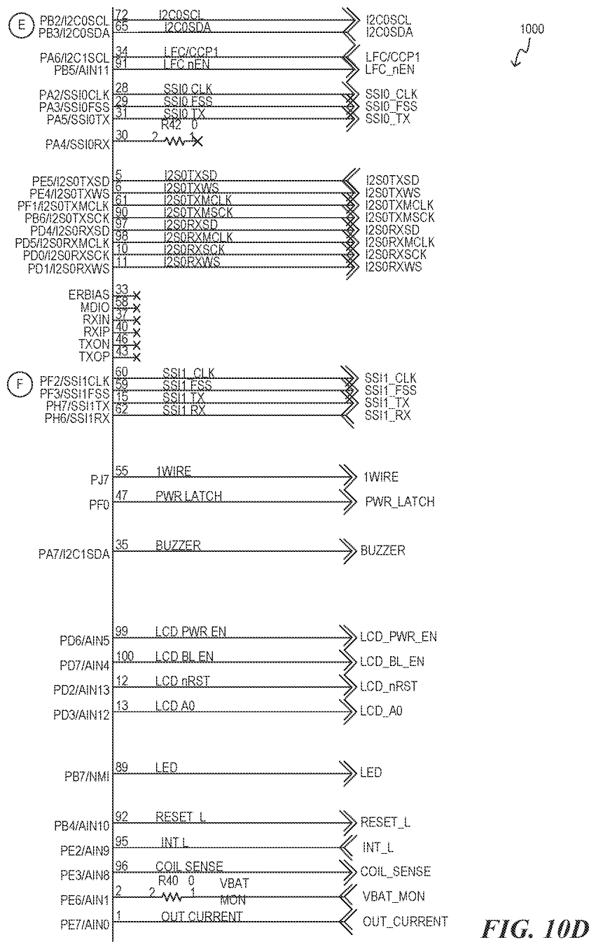

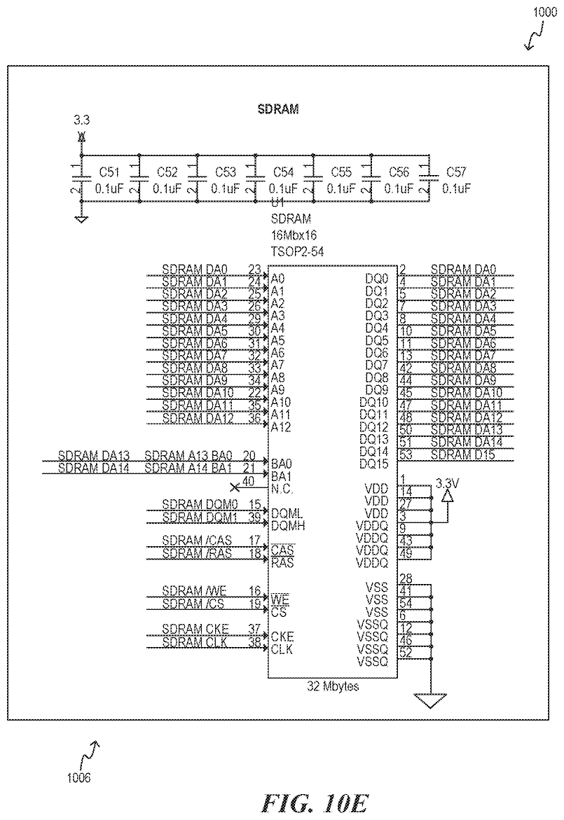

FIGS. 10A-15B illustrate schematics of electronics that the controller 104 may include to perform the various functions described above. The various electronics may be integrated into one or more programmable controllers or may include discrete electronic components electrically and communicatively coupled to each other.

FIGS. 10A-10E illustrate microcontroller circuitry 1000 for operating the controller 104. The circuitry 1000 includes a microprocessor 1002, a reset circuit 1004, and a volatile memory 1006. The microcontroller may be a standard microprocessor, microcontroller or other similar processor, or alternatively be a tamper-resistant processor to improve security. The microprocessor 1002 may include a number of analog and/or digital communication pins to support communications with electronics that are both external and internal to the housing 900. The microprocessor 1002 may include USB pins 1008 to support communication via the USB protocol, display pins 1010 to communicate with a visual interface, and audio pins 1012 to provide an audio interface, in addition to other data communication pins.

Microcontroller 1002 can be configured to use the USB pins 1008 to securely receive cognate files from one or more external devices. Encryption of the cognate file may increase security of the contents of the cognate file. Encryption systems regularly suffer from what is known as the key-distribution-problem. The standard assumption in the cryptographic community is that an attacker will know (or can readily discover) the algorithm for encryption and decryption. The key is all that is needed to decrypt the encrypted file and expose its intellectual property. The legitimate user of the information must have the key. Distribution of the key in a secure way attenuates the key-distribution-problem.

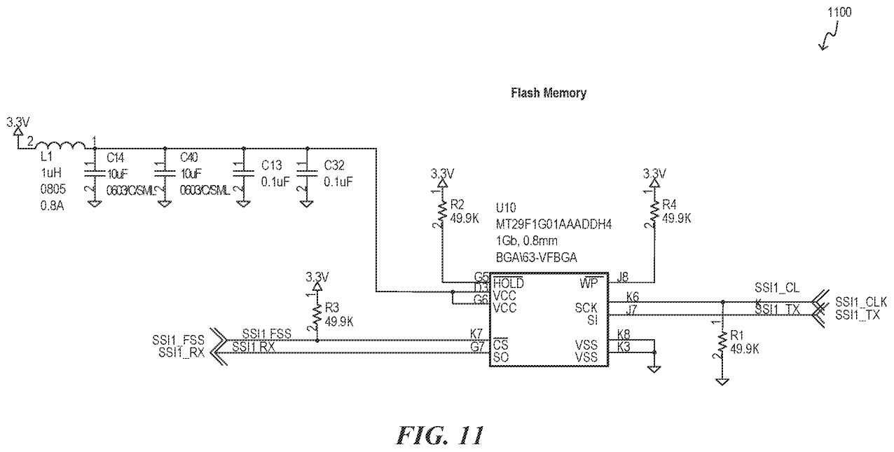

In some embodiments, the microcontroller 1002 is configured to use the Advanced Encryption Standard (AES). AES is a specification for the encryption of electronic data established by the U.S. National Institute of Standards and Technology (NIST) and is used for inter-institutional financial transactions. It is a symmetrical encryption standard (the same key is used for encryption and decryption) and can be secure while the key distribution security is maintained. In some implementations, the microcontroller 1002 uses a 128-bit AES key that is unique to each controller and is stored in non-volatile memory 1100 (illustrated in FIG. 11). The encryption key can be random to reduce the likelihood of forgery, hacking, or reverse engineering. The encryption key can be loaded into non-volatile memory 1100 during the manufacturing process or before the controller is delivered to users. Using AES encryption, the ulRFE® signal can be encrypted and uploaded to one or more servers to facilitate selective delivery to various controllers 104. For example, a medical professional may obtain authorization to download cognates to controllers for his/her application. When the medical professional contacts and logs in to a server to obtain a cognate, the professional may first need to provide some information, e.g., may need to identify the target device (the controller), for the server (e.g., by a globally unique ID (GU ID) stored in the controller) so that the server can look up the target device in a database and provide a cognate file that is encrypted with a key that is compatible with the controller. The encrypted cognate file can then be loaded into the non-volatile memory 1100 via the microcontroller 1002, using USB or another communications protocol. Alternatively, or additionally, the encrypted cognate file may be stored directly to the non-volatile memory 1100 during the manufacturing process to reduce the likelihood of interception of the cognate file, and before the front and back portions of the housing are sealed together.

The microcontroller 1002 can also be configured to log use of the system 100. The log can be stored in a non-volatile memory 1100 and downloaded when a user delivers a controller 104 back to the device distributor, e.g., after the prescribed time allotment for the controller 104 has depleted. The log can be stored in a variety of data formats or files, such as, separated values, as a text file, or as a spreadsheet to enable the display of activity reports for the controller 104. In some implementations, the microcontroller 1002 is configured to log information related to errors associated with coil connections, electrical characteristics of the coil over time, dates and times of use of the system, battery charge durations and discharge traditions, and inductance measurements or other indications of a coil being placed in contact with a human, a mammal, and/or an animal. The microcontroller 1002 can provide log data or the log file to a system monitor using a USB port or other mode of communication to allow the monitor to evaluate the quality and/or function of the system and the quantity and/or use of the system. Notably, the microcontroller 1002 can be configured to log any disruptions in cognate delivery and can log any errors, status messages, or other information provided to the user through user interface of the controller 104 (e.g., using the LCD screen).

The microcontroller 1002 can be configured to use the volatile memory 1006 to protect the content of the cognate file. In some implementations, the cognate file is encrypted when the microcontroller 1002 transfers the file from an external source into non-volatile memory 1100. The microcontroller 1002 can then be configured to only store decrypted versions of the content of the cognate file in volatile memory 1006. By limiting the storage of decrypted content to volatile memory 1006, the microcontroller 1002 and thus the controller 104 can ensure that decrypted content is lost when power is removed from the microcontroller circuitry 1000.

The microcontroller 1002 can be configured to execute additional security measures to reduce the likelihood that an unauthorized user will obtain the contents of the cognate file. For example, the microcontroller 1002 can be configured to only decrypt the cognate file after verifying that an authorized or legitimate coil and cable assembly 102 has been connected to the controller 104. As described above, the coil and cable assembly 102 may include an integrated circuit that may store one or more encrypted or not encrypted identifiers for the coil and cable assembly 102. In some implementations, the microcontroller 1002 is configured to verify that an authorized or prescribed coil and cable assembly 102 is connected to the controller 104. The microcontroller 1002 may verify the authenticity of a coil and cable assembly 102 by comparing the identifier from the integrated circuit of the coil and cable assembly 102 with one or more entries stored in a lookup table in either volatile memory 1006 or non-volatile memory 1100. In other implementations, the microcontroller 1002 may be configured to acquire a serial number of the integrated circuit and measure electrical characteristics of the coil and cable assembly 102 and perform a cryptographic function, such as a hash function, on a combination of the serial number and the electrical characteristics. Doing so may deter or prevent an unauthorized user from copying the contents of the integrated circuit of the coil and cable assembly 102 into a duplicate integrated circuit associated with an unauthorized copy of a coil and cable assembly.

The microcontroller 1002 can be configured to delete the cognate file from volatile memory 1006 and from non-volatile memory 1100 in response to fulfillment of one or more predetermined conditions. For example, the microcontroller 1002 can be configured to delete the cognate file from memory after the controller has delivered the prescribed drug-simulating signals for a specific period of time, e.g., 14 days. In other embodiments, the microcontroller 1002 can be configured to delete the cognate file from memory after the controller detects a coupling of the controller 104 with an unauthorized coil and cable assembly. The microcontroller 1002 can be configured to delete the cognate file after only one coupling with an unauthorized coil and cable assembly, or can be configured to delete the cognate file after a predetermined number of couplings with an unauthorized coil and cable assembly. In some implementations, the microcontroller can be configured to monitor an internal timer and delete the cognate file, for example, one month, two months, or longer after the cognate file has been installed on the controller 104.

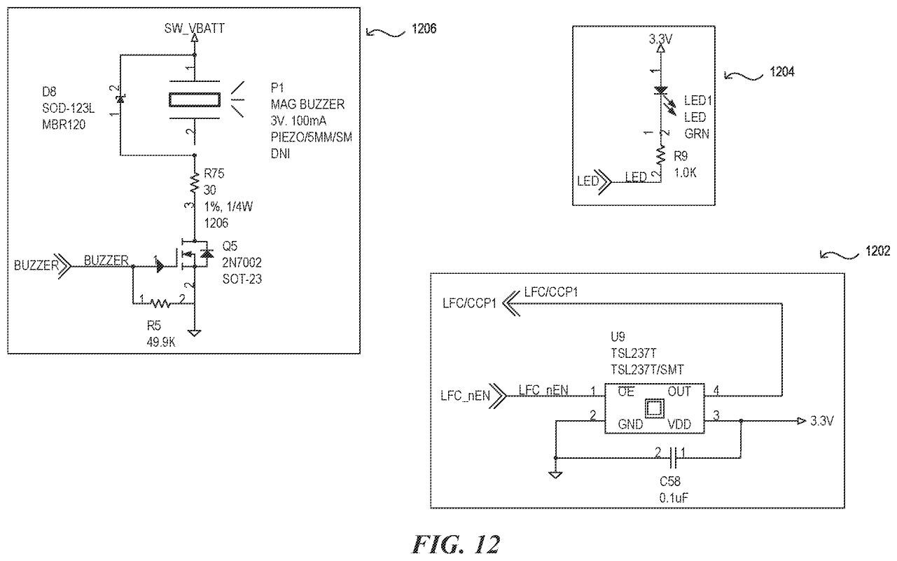

The microcontroller 1002 can be configured to delete the cognate file from volatile memory 1006 and from non-volatile memory 1100 in response to input from one or more sensors. FIG. 12 illustrates a sensor 1202 that may provide a signal to the microcontroller 1002 in response to a physical disruption of the housing 900 of the controller 104. For example, the sensor 1202 can be a light sensor that detects visible and non-visible wavelengths within the electromagnetic spectrum. For example, the sensor 1202 can be configured to detect infrared, visible light, and/or ultraviolet light. Because the detection of light within the housing 900 can be an indication of intrusion into the housing 900, the microcontroller 1002 can be configured to delete and/or corrupt the cognate file upon receipt of a signal from the sensor 1202. In some implementations, the sensor 1202 is a light sensor. In other implementations, the sensor 1202 can be a pressure sensor, a capacitive sensor, a moisture sensor, a temperature sensor, or the like.

In response to detection of unauthorized use of the controller 104, or to increase the user-friendliness of the system 100, the microcontroller 1002 can use various indicators or interfaces to provide information to a user. As examples, FIG. 12 illustrates an LED 1204 and an audible buzzer 1206. The microcontroller 1002 can illuminate the LED 1204 and/or actuate the audible buzzer 1206 in response to user error, unauthorized tampering, or to provide friendly reminders of deviation from scheduled use of the system 100. Although one LED is illustrated in the LED 1204, multiple LEDs having various colors can also be used. Additionally, although the audible buzzer 1206 is described as a buzzer, in other implementations, the audible buzzer 1206 can be a vibrating motor, or a speaker that delivers audible commands to facilitate use of the system 100 by sight impaired users.

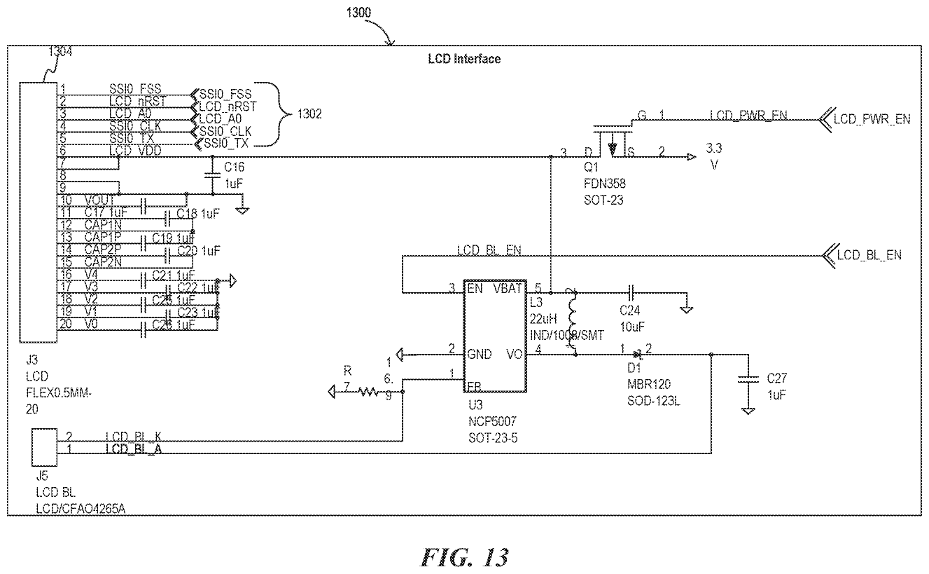

FIG. 13 illustrates an LCD interface 1300 that the microcontroller 1002 can manipulate to interact with a user. The LCD interface 1300 can receive various commands from the microcontroller 1002 at input pins 1302. In response to inputs received from the microcontroller 1002, an LCD screen 1304 can be configured to display various messages to a user. In some implementations, the LCD screen 1304 displays messages regarding battery status, duration of prescription use or exposure, information regarding the type of prescription being administered, error messages, identification of the coil and cable assembly 102, or the like. For example, the LCD screen 1304 can provide a percentage or a time duration of remaining battery power. The LCD screen 1304 can also provide a text-based message that notifies the user that the battery charge is low or that the battery is nearly discharged. The LCD screen 1304 can also be reconfigured to provide a name of a prescription or exposure period (e.g., corresponding name of the physical drug, chemical or other agent) and/or a human, a mammal, and/or an animal part for which the prescription or exposure is to be used. The LCD screen 1304 can also provide notification of elapsed-time or remaining-time for administration of a prescription or exposure. If no additional prescription or exposure time is authorized, the LCD screen 1304 can notify the user to contact the applicable prescriber or provider.

The LCD screen 1304 can be configured to continuously or periodically provide indications regarding the status of the connection between a coil and the controller. In some implementations, the LCD screen 1304 can be configured to display statuses or instructions such as, “coil connected,” “coil not connected,” “coil identified,” “unrecognized coil,” “reconnect coil,” or the like. In some implementations, the LCD screen 1304 can provide a graphical representation of a coil and flash the coil when the coil is connected properly or improperly. Alternatively, or additionally, the controller can monitor an impedance from the coil to detect a change, a possible removal, or loss of the coil from the area to be treated, and provide a corresponding error message. The LCD interface 1300, in other implementations, can be a touch screen that delivers information to the user in addition to receiving instructions or commands from the user. In some implementations, the microcontroller 1002 can be configured to receive input from hardware buttons and switches to, for example, power on or power off the controller 104. The switch on the device permits an on-off nature of treatment so that treatment may selectively be switched on and off if needed.

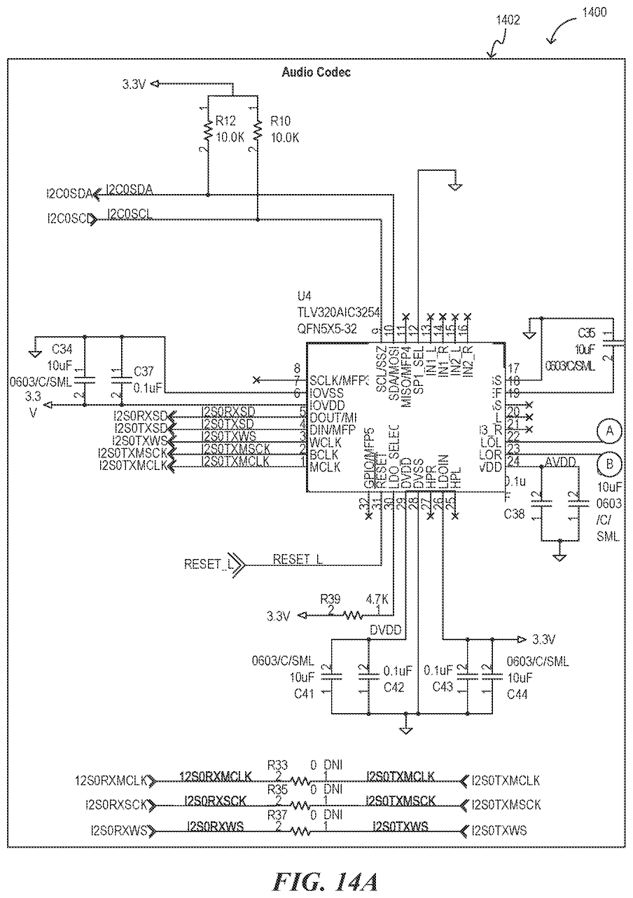

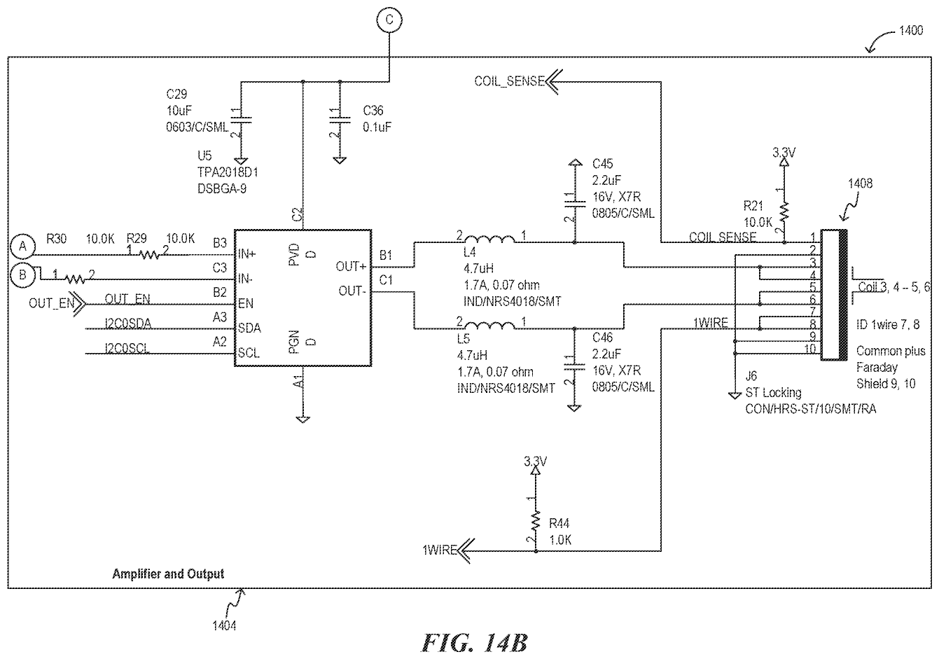

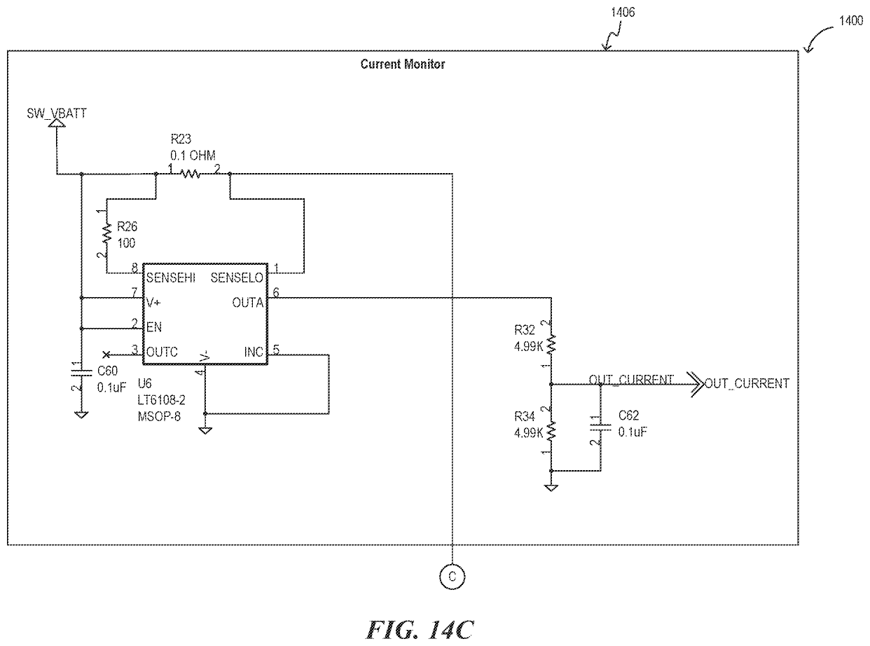

FIGS. 14A-14C illustrate signal generation circuitry 1400 that may be used to drive the coil and cable assembly 102 with drug or chemical-simulating signals. The circuitry 1400 may include an audio coder-decoder 1402, and output amplifier 1404, and a current monitor 1406. The audio coder-decoder 1402 may be used to convert digital inputs received from volatile memory 1006, non-volatile memory 1100, or from microcontroller 1002 into analog output signals useful for driving the coil and cable assembly 102. The audio coder-decoder 1402 may be configured to output the analog output signals to the output amplifier 1404. In some implementations, the output amplifier 1404 is programmable so that the intensity or amplitude of the signals transmitted to the coil may be varied according to the treatment prescribed for the human, mammal, and/or animal.

Because the controller 104 can be connected with coils having different sizes, shapes, and numbers of windings, the output amplifier 1404 can be configured to adjust the intensity level of the ulRFE® cognates delivered to the coil so that each coil delivers a drug or chemical-simulating ulRFE® cognate that is uniform between different coils, or different between coils, for a particular prescription or exposure period. The coil dimensions and electrical characteristics influence the depth and breadth of the magnetic field, so programmatically adjusting the output intensity of the output amplifier 1404 to deliver uniform drug or chemical-simulating ulRFE® cognates can advantageously enable the selection of a coil that is appropriate for a particular treatment area, to avoid inadvertently altering the prescription or exposure period. As described above, the controller 104 can determine the dimensions and electrical characteristics of a coil by reading such information from the integrated circuit 610 (shown in FIGS. 6 and 7). The cognate generation circuitry 1400 can be configured to use the dimensional and electrical characteristic information acquired from the coil to programmatically adjust the level of intensity of ulRFE® output by the output amplifier 1404.

The output amplifier 1404 may include a low pass filter that significantly reduces or eliminates ulRFE® output having a frequency higher than, for example, 50 kHz. In other implementations, the low pass filter can be configured to significantly reduce or eliminate ulRFE® output having a frequency higher than 22 kHz. The cognate generation circuitry 1400 may use the current monitor 1406 to determine electrical characteristics of the coil and cable assembly 102 and/or to verify that ulRFE® output levels remain within specified thresholds. The ulRFE® cognate generation circuitry 1400 may also include a connector 1408 that mates with the connector 206 of the coil and cable assembly 102. The connector 1408 can provide the electrical interface between the microcontroller 1002 and the coil and cable assembly 102.

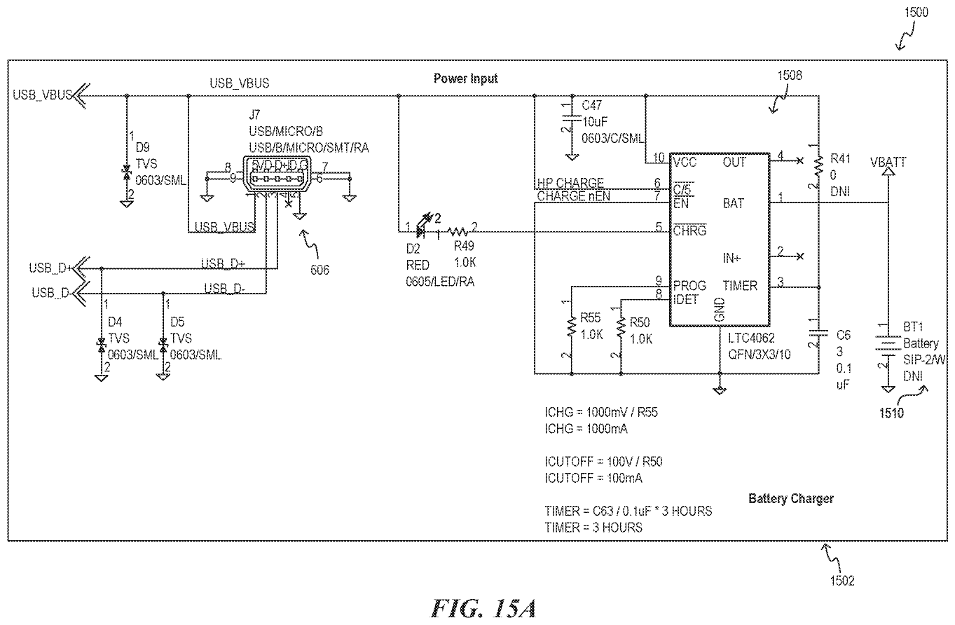

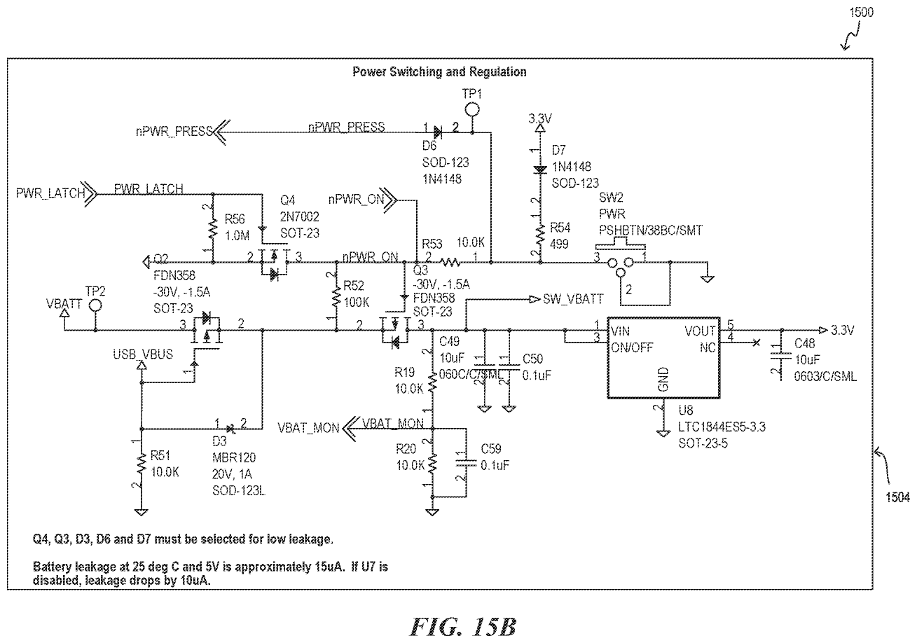

FIGS. 15A-15B illustrate power control circuitry 1500 for receiving and regulating power to the controller 104. The power control circuitry 1500 includes power input circuitry 1502 and power regulation circuitry 1504. The power input circuitry 1502 can include a connector 1506, e.g., a micro-USB connector, to receive power from an external source for recharging a battery 1510. The power input circuitry 1502 can also include a charging circuit 1508 that monitors a voltage level of the battery 1510 and electrically decouples the battery from the connector 1506 when the battery 1510 is sufficiently charged. The power regulation circuitry 1504 can be used to convert a voltage level of the battery 1510 to a lower voltage for use by the various circuits of the controller 102. For example, when fully charged, the battery 1510 may have a voltage of about 4.2 to 5 volts, whereas the microcontroller may have an upper voltage threshold of 3.5 volts. The power regulation circuitry 1504 can be configured to convert the higher voltage of the battery, e.g., 4.2 volts, to a lower voltage, e.g., 3.3 volts, that is usable by the electronic devices of the controller 104.

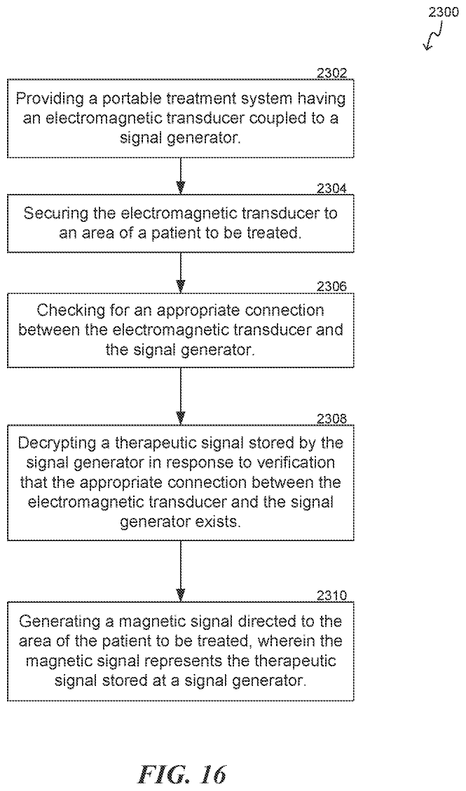

FIG. 16 illustrates a method 1600 of operating a portable system that may be used to provide magnetic field treatment that is non-invasive, non-thermal, non-ionizing and mobile.

At block 1602 an electromagnetic transducer is coupled to a ulRFE® cognate generator. The electromagnetic transducer can be a coil having various shapes and sizes according to the size of the object or condition to be treated.

At block 1604 the electromagnetic transducer is secured to an area of the animal to be treated. The transducer may be secured using elastic bandages, gauze, tape, or the like.

At block 1606, the ulRFE® cognate generator checks for an appropriate connection to the electromagnetic transducer. The ulRFE® cognate generator can be configured to verify an identification or electrical characteristics of the electromagnetic transducer, such as a resistance or impedance of the transducer to ensure that an appropriate transducer is coupled to the generator. In some implementations, the ulRFE® cognate generator can be configured to periodically monitor the electrical characteristics of the electromagnetic transducer to ensure that an appropriate connection is maintained. For example, if the ulRFE® cognate generator detects an increase in resistance or decrease in inductance, the ulRFE® cognate generator may be configured to cease delivery of ulRFE® to the electromagnetic transducer. The ulRFE® cognate generator may cease delivery of ulRFE® when unexpected electrical characteristics are detected to protect the health and/or safety of the subject or to protect the subject being treated, and to prevent unauthorized attempts to acquire generated ulRFE® cognates. As discussed above, the ulRFE® cognate generator may be configured to log the periodic checks of the electrical characteristics of the electromagnetic transducer and can provide the log data for review. Other security checks may be performed as described herein.

At block 1608 the ulRFE® cognate generator decrypts a ulRFE® cognate stored by the ulRFE® cognate generator in response to verification that an appropriate connection between the electromagnetic transducer and the ulRFE® cognate generator exists. Where the term “ulRFE® cognate” is used herein, the term generally applies to any stored cognate that the disclosed system uses to induce a chemical, biological or other change in a biological system.

At block 1610 the electromagnetic transducer generates a ulRFE® cognate directed to the human, mammal, and/or animal or specific anatomical region of the human, mammal, and/or animal to be treated. The cognate used to generate the specific electromagnetic field is stored in the ulRFE® cognate generator. According to various implementations, the cognate's magnetic field has a frequency in the range of 0 Hz to 22 kHz.

In some instances, the ulRFE® cognate can be delivered to a subject (e.g., human, mammal, and/or animal) in addition to administering a drug, chemical or other agent to the subject. For example, the drug, chemical or other agent can be administered and/or applied to human, mammal, and/or animal, or area of the human, mammal, and/or animal to be treated with the ulRFE® cognate before or after the ulRFE® cognate is delivered to the subject. In some instances, the ulRFE® cognate is derived from a sample of the same drug, chemical or other agent administered to the subject. In other instances, the ulRFE® cognate derived from a sample of a different drug, chemical or other agent than that administered to the subject. Moreover, the drug, chemical or other agent and/or the ulRFE® cognate can be delivered to the subject more than once and in any sequence, for example, drug, chemical or other agent+ulRFE® cognate+drug, chemical or other agent, or ulRFE® cognate+drug, chemical or other agent+ulRFE® cognate, etc. In further instances, the sequences can include more than one ulRFE® cognate and more than one drug, chemical or other agent.

In some implementations, the cognate of a sample of a drug, chemical or other agent may be acquired by placing the sample in an electromagnetic shielding structure and by placing the sample proximal to, at least one, superconducting quantum interference device (SQUID) (or magnetometer). The drug, chemical or other agent sample is placed in a container having both magnetic and electromagnetic shielding, where the sample drug, chemical or other agent acts as a signal source to record the ulRFE® molecular cognate. In some embodiments, noise is injected into the drug, chemical or other sample at a noise amplitude sufficient to generate stochastic resonance, where the noise has a substantially uniform amplitude over multiple frequencies. The stochastic resonance induced by noise injection may allow an otherwise undetectable signal to be recorded. Using the superconducting quantum interference device (SQUID) (or the magnetometer), the electrostatic surface potential of the drug, chemical or other agent sample is detected and recorded as an electromagnetic time-domain signal composed of sample-source radiation superimposed on the injected noise (if any). The recording of an electromagnetic time-domain signal from a sample may be repeated at multiple noise levels to enable the detection of a sample-specific signal. In some embodiments, the cognate of a sample of a drug, chemical or other agent may be acquired by dissolving the sample in a solvent, e.g., water, dimethyl sulfoxide (DMSO) or ethanol and placing the sample proximal to, at least one, SQUID (or magnetometer).

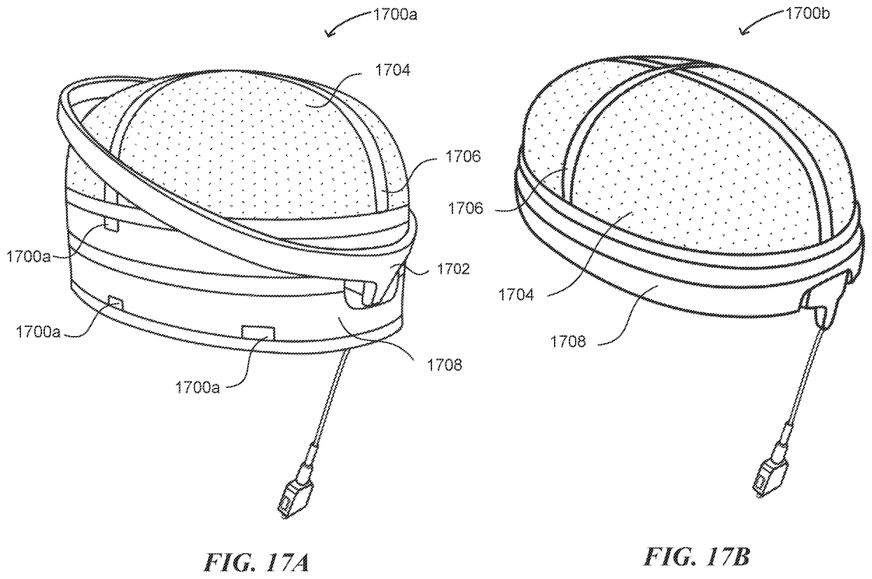

FIGS. 17A and 17B illustrate example embodiments of headgear 1700 (inclusive of 1700a and 1700b) that may be used to position or secure a coil 1702 around the cranium of an animal. The headgear can include a breathable mesh 1704, elastic straps 1706, and a band 1708. The breathable mesh 1704, elastic straps 1706, and the band 1708 can provide a comfortable apparatus for carrying, securing, or otherwise positioning the coil 1702 around the cranium of an animal. The headgear 1700 may also include fasteners 1710 (inclusive of 1710a, 1710b, 1710c) for securing the band 1708 over the coil 1702. The fasteners 1710 may be influenced with Velcro, snaps, or other types of securing devices. In FIG. 17A, the headgear 1700a illustrates the coil 1702 in an exposed or unsecured position. In FIG. 17B, the headgear 1700b illustrates the coil 1702 in a secured position.

In some embodiments, systems and methods disclosed herein can include other types of apparatuses to position or secure one or more coils relative to a subject. For example, the coil can be carried or otherwise supported by pads of various shapes and sized to deliver the signal to the subject at a particular site of the subject's body experiencing pain. For example, the pad can be a back pad, a neck pad, an arm pad, a leg pad, a pad that extends along the subject's entire body (e.g., a mat, mattress pad), and/or other type of pad sized and shaped to carry the coil and target a specific portion of the subject's body. In some configurations, the pad or a portion thereof is flexible so as to conform to a shape of at least a portion of the subject's body when positioned thereon (e.g., when the subject lays or otherwise rests a portion of the subject's body on the pad, when the pad is attached to or otherwise put in close contact with a portion of the pad). In other configurations, the pad or a portion thereof is stiff so as to provide support to at least a portion of the subject's body.

### EXAMPLES

The following examples are illustrative of several embodiments of the present technology using the EMulate Voyager ulRFE® system, a non-invasive device for delivery of a ulRFE® signal for reducing pain and/or providing an analgesic effect. “Signals and Associated Methods”, “System Design”, and “Animals” were used in each of the examples.

### Signals and Associated Methods

In the following examples, the EMulate Voyager ulRFE® system was studied in five animal pan model studies to assess safety and feasibility of the system for delivery of one or more of twelve different ulRFE® signals for reducing pain and/or providing an analgesic effect. Twelve cognates were generated, WAV 1-WAV 6 and WAV 1′ WAV 6′, each WAV cognate derived from one or more pharmacological compounds in solution. WAV 1-WAV 6 (also referred to herein as original signals) were derived directly from the dynamic magnetic field of one or more pharmacological compounds and comprise signal frequencies ranging from DC to 22 kHz. WAV 1′-WAV 6′ (also referred to herein as modified signals) were derived from WAV 1-WAV 6 through a resampling process. The modified signals (WAV 1′-WAV 6′) each comprise signal frequencies ranging from DC to 6 kHz. Exemplary cognates, signal frequencies, and pharmacological compounds from which they were derived are provided in Table 1.

TABLE 1
Cognates, signal frequencies, and corresponding
pharmacological compounds

| | | Pharmacological |
|---|---|---|
| Cognate | Frequency | compound |
| WAV 1 | DC to 22 kHz | Fentanyl |
| WAV 2 | DC to 22 kHz | Cannabidiol |
| WAV 3 | DC to 22 kHz | Naproxen |
| WAV 4 | DC to 22 kHz | Hydromorphone |
| WAV 5 | DC to 22 kHz | Dexamethasone |
| WAV 6 | DC to 22 kHz | Indomethacin |
| WAV 1′ | DC to 6 kHz | Fentanyl |
| WAV 2′ | DC to 6 kHz | Cannabidiol |
| WAV 3′ | DC to 6 kHz | Naproxen |
| WAV 4′ | DC to 6 kHz | Hydromorphone |
| WAV 5′ | DC to 6 kHz | Dexamethasone |
| WA 6′ | DC to 6 kHz | Indomethacin |

A broad banded, white noise control signal (DC—22 kHz) was generated using a Stanford Research Instruments Arbitrary Waveform Generator (Stanford Research Systems, Sunnyvale CA) and stored as a WAV file. The White Noise signal was used as a general magnetic field control signal.

### System Design

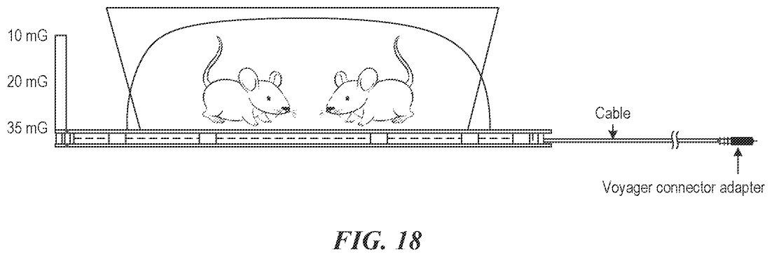

The EMulate Voyager system was configured to be able to transmit the recorded signals described above to rats. As shown in FIG. 18, the EMulate Voyager system was placed directly underneath a rat cage. The magnetic field envelope of the transmitted signals extended above the surface of the EMulate Voyager system. The magnetic field AC root-mean square (RMS) flux density at the surface of the EMulate Voyager system was in the ˜30-70 mG range, depending on the signal being emitted.

### Animals

All the animal models were validated, with a long-standing record of use and well characterized pathways. An initial high through-put screen (the ALGOGram™) was used to measure the pain reducing potential of select signals to determine whether an analgesic/anti-inflammatory effect is detected (data not shown). Signals that showed an effect were selected for confirmatory testing powered to detect a statistically significant effect.

Male Sprague-Dawley rats (SPF status, Janvier, France) were used for each exposure to a WAV file emitted as magnetic field (24 hours of constant magnetic field exposure prior to testing) for each test in the ALGOGram™ screen and the second, confirmatory pain assays. Rats were housed in a temperature (20-24° C.) and relative humidity (45%-65%) controlled room and acclimated to an artificial day/night cycle of 12 hours light/dark. Rats had free access to water and were fed ad libitum. Animals were housed two or four per cage (type E and type III H) during their acclimation period and one or two per cage type 2150E (18 cm×30 cm) during the 24 hours of exposure to the magnetic field.

### Example 1. Reducing Pain in and/or Providing an Analgesic Effect to Cold Allodynia Using ulRFE® Signal

This example demonstrates that the EMulate Voyager system is feasible and safe for the reduction of pain and/or providing an analgesic effect against cold allodynia. The therapy was delivered non-invasively, and no serious adverse events attributed to the therapy were reported.

As described above, systems configured in accordance with the present technology can record the dynamic magnetic field of one or more molecules in solution (e.g., water, dimethyl sulfoxide (DMS) or ethanol). One or more of these systems can transmit the recorded magnetic field information (e.g., ulRFE®, cognate, signal, etc.) to an in vitro or in vivo system, such as cells or a living subject safely. The EMulate Voyager ulRFE® system, a non-invasive device, was studied in a rat feasibility study to assess safety and feasibility of the treatment for peripheral neuropathy.

In the present example, the Voyager system was used to administer ulRFE® to a group of rats in a cold water immersion test (Cheng et al., Front Pharmacol. 8:344 (2017)). A total of 90 rats were used in the present example: 10 were exposed to MC 1%; 10 were exposed to White Noise; 10 were exposed to WAV 1; 10 were exposed to WAV 1′; 10 were exposed to WAV 2; 10 were exposed to WAV 2′; 10 were exposed to WAV 3; 10 were exposed to WAV 3′; and 10 received 100 mg/kg duloxetine (positive control).

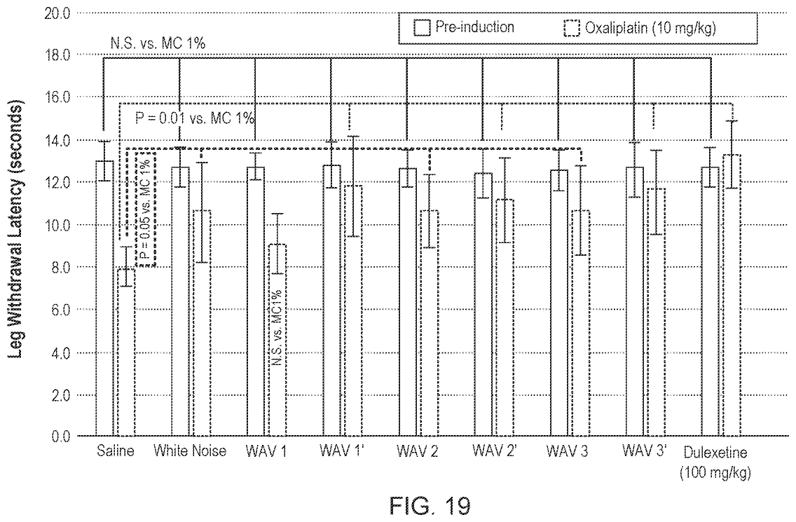

Acute peripheral neuropathy was induced by a single intraperitoneal injection of oxaliplatin (10 mg/kg) 72 hours before performing a paw immersion test to assess cold allodynia. After oxaliplatin injection, rats were exposed to a magnetic field for 24 hours before the cold immersion test was performed. In the paw immersion test, the latency of hind paw withdrawal was measured after immersion of the hind paw in a temperature-controlled water-bath (cryothermostat) with a temperature fixed at 10° C. (±0.5° C.). The paw immersion test was performed on each rat pre-induction of cold allodynia with oxaliplatin and post induction with oxaliplatin. Signal exposed groups were compared to the MC 1% group (negative control) using Dunnett's test (Groups=8, N=10 per Group, α=0.05) after significant one-way ANOVA. Results are shown in FIG. 19.

As shown in FIG. 19, a single intraperitoneal administration of Oxaliplatin (10 mg/kg) induced cold allodynia as evidenced by a marked and significant decrease in the paw withdrawal latency 72 hours after injection in the Oxaliplatin/MC-treated group as compared to the pre-induction baseline (P<0.01 as compared to the pre-induction baseline of the corresponding group, Wilcoxon test). A Dunnett's test comparing each signal exposure group to the MC 1% group (negative control) demonstrated no significant differences between groups (average time to leg withdrawal: 12.7 seconds) before induction of cold allodynia via oxaliplatin injection. Post-oxaliplatin induction, the duloxetine drug group (positive control) significantly increased the leg withdrawal time (13.2±1.6 seconds) compared to the MC 1% control group (7.9±1.0 seconds). All three modified signals (WAV 1′; WAV 2′; WAV 3′) demonstrated a significant increase in leg withdrawal delay (11.8±2.3 sec; 11.2±1.9 sec; 11.7±1.8 sec, respectively) versus the MC 1% control group (7.9±1.0 seconds; P=0.01). Two unmodified signals (WAV 2 and WAV 3) also demonstrated significantly increased leg withdrawal delays (10.7±1.7 and 10.7±2.1 seconds, respectively) at P=0.05 versus the MC 1% control group. The White Noise signal achieved a statistically significant effect (10.6±2.3 seconds; P=0.05) versus the MC 1% control group. The WAV 1 signal did not reach a statistically significant delay in leg withdrawal (9.1±1.5 seconds; Not Significant, N.S.) versus the MC 1% control group.

### Example 2. Reducing Pain in and/or Providing an Analgesic Effect to Mechanical Hyperalgesia Using ulRFE® Signal

This example demonstrates that the EMulate Voyager system is feasible and safe for the reduction of pain and/or providing an analgesic effect against mechanical hyperalgesia. The therapy was delivered non-invasively and no serious adverse events attributed to the therapy were reported.

As described above, systems configured in accordance with the present technology can record the dynamic magnetic field of one or more molecules in solution (e.g., water, dimethyl sulfoxide (DMS) or ethanol). One or more of these systems can transmit the recorded magnetic field information (e.g., ulRFE®, cognate, signal, etc.) to an in vitro or in vivo system, such as cells or a living subject safely. The EMulate Voyager ulRFE® system, a non-invasive device, was studied in a rat feasibility study to assess safety and feasibility of the treatment for inflammatory pain.

In the present example, the Voyager system was used to administer ulRFE® to a group of rats in a mechanical hyperalgesia test (Randall et al., Arch Int Pharmacodyn Ther. 111:409-19 (1957)). A total of 90 rats were used in the present example: 10 were exposed to MC 1%; 10 were exposed to White Noise; 10 were exposed to WAV 2; 10 were exposed to WAV 2′; 10 were exposed to WAV 1; 10 were exposed to WAV 1′; 10 were exposed to WAV 4; 10 were exposed to WAV 4′; and 10 received 30 mg/kg Indomethacin (positive control).

Three hours before assessment of the nociceptive threshold using the paw pressure test, 100 μL of a 2% carrageenan suspension was injected into the plantar aspect of the right hind paw of the rat. After carrageenan injection, rats were returned to their cages. Rats assigned to a magnetic field exposure were placed back into the magnetic field.

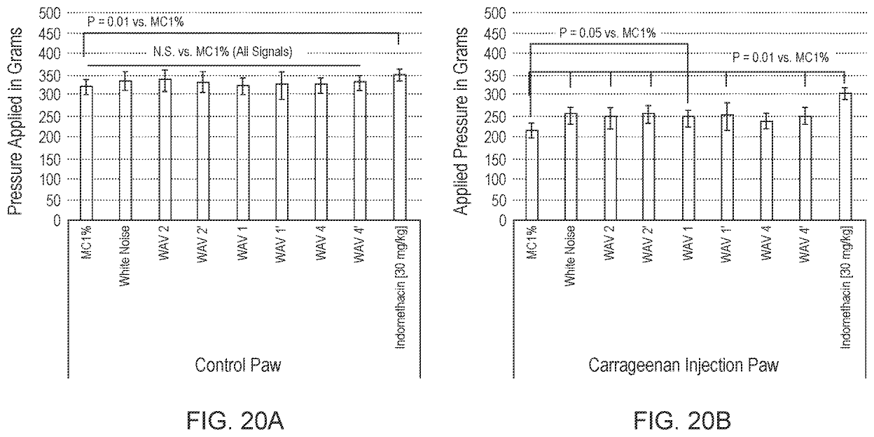

Static mechanical hyperalgesia was assessed using the paw pressure test (Randall & Selitto test). In the paw pressure test, an apparatus is used to steadily exert increasing pressure on the hind paw placed between a flat surface and a blunt pointer of the apparatus. The test was performed on rats with one hind paw inflamed by an injection, and one normal hind paw. Reaction threshold was determined as the pressure (g) required to elicit paw withdrawal and/or vocalization. In the experiment, static mechanical hyperalgesia was assessed twice for both hind paws and the validity of mechanical hyperalgesia was assessed by comparing the control paw and the injured paw using Mann-Whitney rank sum test. The signal and physical drug groups were compared to the MC 1% group and analyzed using Dunnett's test (Groups=9, N=10 per Group, α=0.05) after significant one-way ANOVA. Results are shown in FIGS. 20A-20B.

FIG. 20A shows the amount of pressure (in grams) applied to the left paw (uninjured paw), which was not injected with carrageenan, before it was lifted away from the pressure source (control group) and FIG. 20B shows the amount of pressure applied to the right paw (injured paw), which was injected with carrageenan, before it was lifted away from the pressure source (treatment group). A Dunnett's test comparing the signal exposed groups to the MC 1% group, demonstrated no significant differences between groups in the uninjured paws, except in the Indomethacin injected group (FIG. 20A). Measurements in the carrageenan injected paws demonstrated that WAV 2′, WAV 1′ and WAV 4′ (256±16 grams, 254±16 grams, 252±21 grams, respectively), Indomethacin (306±21 grams) and White Noise (256±18 grams) resulted in a statistically significant difference in applied pressure when compared to the MC 1% group (218±18 grams; P=0.01). The WAV 1 signal (248±19 grams) reached a statistically significant difference against the MC 1% group at P=0.05 level. The WAV 4 signal did not reach a statistically significant difference in applied pressure (240±24 grams; Not Significant, N.S.) versus the MC 1% control group.

### Example 3. Reducing Pain in and/or Providing an Analgesic Effect to Colonic Sensitivity Using ulRFE® Signal

This example demonstrates that the EMulate Voyager system is feasible and safe for the reduction of pain and/or providing an analgesic effect against colonic sensitivity. The therapy was delivered non-invasively, and no serious adverse events attributed to the therapy were reported.

As described above, systems configured in accordance with the present technology can record the dynamic magnetic field of one or more molecules in solution (e.g., water, dimethyl sulfoxide (DMS) or ethanol). One or more of these systems can transmit the recorded magnetic field information (e.g., ulRFE®, cognate, signal, etc.) to an in vitro or in vivo system, such as cells or a living subject safely. The EMulate Voyager ulRFE® system, a non-invasive device, was studied in a rat feasibility study to assess safety and feasibility of the treatment for visceral pain.

In the present example, the Voyager system was used to administer ulRFE® to a group of rats in a colonic sensitivity test (Diop et al., J Pharmacol Exp Ther. 302:1013-22(2002)). A total of 80 rats were used in the present example: 10 received saline and no surgery; 10 received saline and surgery; 10 were exposed to White Noise; 10 were exposed to WAV 1; 10 were exposed to WAV 1′; 10 were exposed to WAV 4; 10 were exposed to WAV 4′; and 10 received 1 mg/kg (−)U50,488H (positive control).

Colonic sensitivity was induced by surgical administration of 2,4,6-trinitrobenzenesulfonic acid solution (TNBS) seven days before behavioral testing (D₋₇). Animals were fasted overnight prior to surgery. Animals were anesthetized by injection of xylazine 10 mg/kg/Ketamine 60 mg/kg, then the colon was exposed through a small incision of the abdomen. TNBS (50 mg/kg, 1 ml/kg) was injected into the proximal part of the colon (1 cm from the caecum). After surgery, animals were returned to their home cages in a regulated environment, and were fed ad libitum until D₋₁, at which point the animals were fasted.

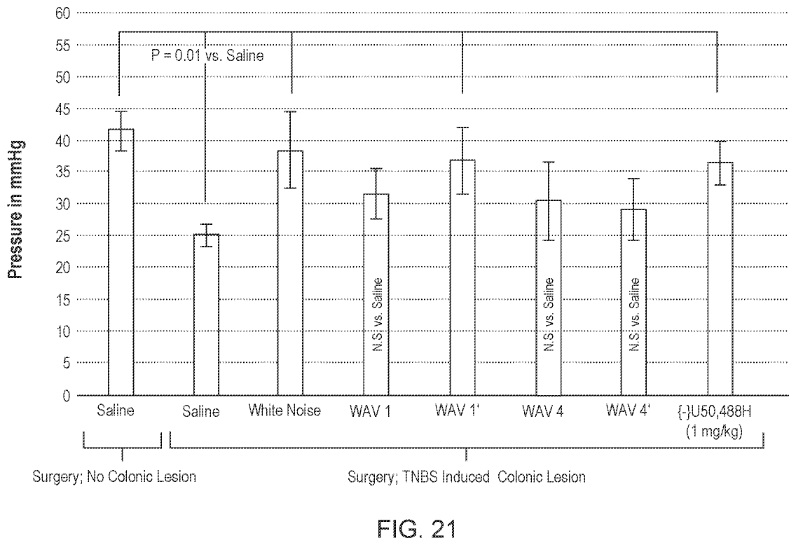

Seven days (D₀) after TNBS injection, colonic sensitivity was assessed on fasted (overnight) animals by measuring the intracolonic pressure required to induce a behavioral response during colonic distension. To perform distension, a 5-cm long balloon was gently inserted into the colon of animals at 10 cm from the anus and the catheter was taped to the base of the tail. After a 30-minute acclimation period with the inserted balloon, colonic pressure was gradually increased by 5 mmHg increments every 30 seconds from 5 to 75 mmHg until pain behavior was evidenced. Pain behavior was characterized by an elevation of the hind part of the animal body and a clearly visible abdominal contraction corresponding to severe cramp. Two determinations were performed at 30 minutes and at 50 minutes. Signal and physical drug treated groups were compared to the Saline (Surgery; TNBS Induced Colonic Lesion) using Dunnett's test (Groups=8, N=10 per Group, α=0.05) after significant One-Way ANOVA. Results are shown in FIG. 21.

A Dunnett's test versus the TNBS lesion, saline group (25±2 mmHg), demonstrated a significant difference between the WAV 1′ (37±5 mmHg) and White Noise (39±6 mmHg) signals and the drug compound (−)U50,488H (37±3 mmHg) at a level of P=0.01. The sham lesioned control rats (saline, no colonic lesion; 42±3 mmHg) that served as a baseline group, reached a significance level of P=0.01. The WAV 1, WAV 4, and WAV 4′ signals (32±4 mmHg, 31±6 mmHg, 29±6 mmHg, respectively) did not reach a statistically significant difference in applied pressure versus the TNBS lesion—saline control group (25±2 mmHg).

### Example 4. Reducing Pain in and/or Providing an Analgesic Effect to Peripheral Mononeuropathy Using ulRFE® Signal

This example demonstrates that the EMulate Voyager system is feasible and safe for the reduction of pain and/or providing an analgesic effect against mononeuropathy. The therapy was delivered non-invasively, and no serious adverse events attributed to the therapy were reported.

As described above, systems configured in accordance with the present technology can record the dynamic magnetic field of one or more molecules in solution (e.g., water, dimethyl sulfoxide (DMS) or ethanol). One or more of these systems can transmit the recorded magnetic field information (e.g., ulRFE®, cognate, signal, etc.) to an in vitro or in vivo system, such as cells or a living subject safely. The EMulate Voyager ulRFE® system, a non-invasive device, was studied in a rat feasibility study to assess safety and feasibility of the treatment for peripheral mononeuropathy.

In the present example, the Voyager system was used to administer ulRFE® to a group of rats in a peripheral mononeuropathy test (Bennett et al., Pain. 33:87-107 (1988)). A total of 50 rats were used in the present example: 10 were exposed to MC 1%; 10 were exposed to White Noise; 10 were exposed to WAV 2; 10 were exposed to WAV 2′; and 10 received 3 mg/kg morphine (positive control).

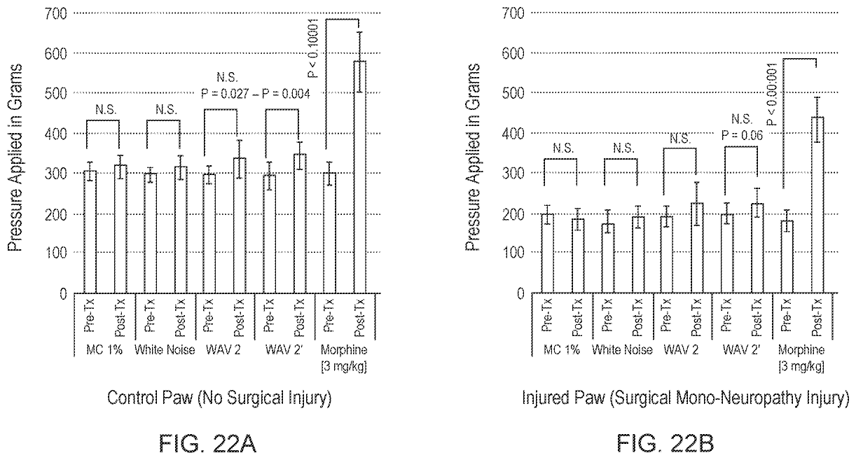

Peripheral mononeuropathy was induced by loose ligation of the sciatic nerve in anaesthetized rats (Xylazine 10 mg/kg i.p., Ketamine 60 mg/kg i.p.) on D₋₁₄. Briefly, the common sciatic nerve was exposed at the level of the middle of the thigh by blunt dissection through the biceps femoris. Proximal to the sciatic trifurcation, four ligatures were tied loosely around it with about 1-mm spacing. Great care was taken to tie the ligatures such that the diameter of the nerve was seen to be just barely constricted. After surgery, animals were allowed to recover for 4 days. Then, the tactile allodynia of the injured paw and the uninjured paw of rats was assessed using the electronic Von Frey. The effect of injury induction was assessed by comparing the control paw to the injured paw, using Bonferroni's test after a significant two-way ANOVA (P<0.001). A pair-wise comparison of pre-treatment versus post-treatment groups (control paw versus control paw; injured paw versus injured paw) using Students T-test (α=0.01) was performed. Results are shown in FIGS. 22A-22B.

FIG. 22A shows the average force applied to the control paw of rats exposed to treatment before and after the applied treatment and FIG. 20B shows the average force applied to the injured paw of rats before and after treatment (30 minutes after morphine/24 hours after signal exposure). A one-way ANOVA within the Pre-Tx control paw group and the Pre-Tx injured paw group did not reach statistical significance. As shown in FIG. 22A, tests on the control paw group (Pre-Tx vs. Post-Tx) resulted in the WAV 2′ (292.8±36 grams vs. 244±34 grams, P=0.004) and the morphine (300±30 grams vs. 578±73 grams, P<0.00001) positive control group reaching a statistically significant difference in applied pressure. None of the other control paw groups (MC1%, White Noise and WAV 2) reached a statistically significant difference. In the injured paw group, shown in FIG. 22B, only the morphine treated injured paw group, reached statistical significance Pre-Tx vs. Post-Tx (180±27 grams vs. 438±51 grams). None of the other control paw groups (MC 1%, White Noise, WAV 2, and WAV 2′) reached a statistically significant difference.

### Example 5. Reducing Pain in and/or Providing an Analgesic Effect to Abdominal Contractions Using ulRFE® Signal

This example demonstrates that the EMulate Voyager system is feasible and safe for the reduction of pain and/or providing an analgesic effect against abdominal contractions. The therapy was delivered non-invasively, and no serious adverse events attributed to the therapy were reported.

As described above, systems configured in accordance with the present technology can record the dynamic magnetic field of one or more molecules in solution (e.g., water, dimethyl sulfoxide (DMS) or ethanol). One or more of these systems can transmit the recorded magnetic field information (e.g., ulRFE®, cognate, signal, etc.) to an in vitro or in vivo system, such as cells or a living subject safely. The EMulate Voyager ulRFE® system, a non-invasive device, was studied in a rat feasibility study to assess safety and feasibility of the treatment for visceral pain.

In the present example, the Voyager system was used to administer ulRFE® to a group of rats in an acetic acid writhing test (Koster et al., Federation Proceedings. 18:412-8 (1959)). A total of 90 rats were used in the present example: 10 received saline; 10 were exposed to White Noise; 10 were exposed to WAV 4; 10 were exposed to WAV 4′; 10 were exposed to WAV 5; 10 were exposed to WAV 5′; 10 were exposed to WAV 6; 10 were exposed to WAV 6′; and 10 were exposed to 1 mg/kg (−)U50,488H (positive control).

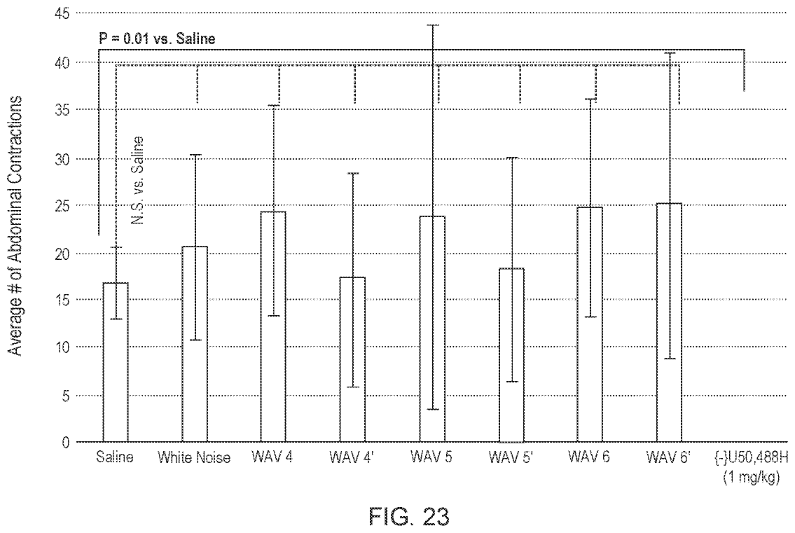

Abdominal contraction was induced by intraperitoneal injection of 0.6% acetic acid solution in rats (10 mL/kg) and recorded. The number of contractions were recorded from 5 to 15 minutes after injection of the solution. Signal exposed groups were compared to the saline group using Dunnett's test after significant one-way ANOVA. Results are shown in FIG. 23. As shown in FIG. 23, the positive control, (−)U50,488, was the only treatment that reached a statistically significant effect.

Signals tested, statistical results and tests used in the present examples are displayed in Table 2. All statistical comparisons were post-hoc and alpha levels for significance were set at α=0.05, apart from the Bennet Model of Peripheral Mononeuropathy (P=0.01). Each treatment/exposure arm had N=10 rats and all statistical comparisons were made against the saline or MC 1% control group arm for the pain model tested, apart from the Bennet Model of Peripheral Mononeuropathy assay. All rats in the pain model groups had a known pain inhibiting positive control arm (either duloxetine, indomethacin, (−)U50,488H, or morphine).

TABLE 2
Outcomes of Pain Models described in Examples 1-5 and post-hoc statistical
analysis of results after ANOVA

| | | | | Bennett | | |
|---|---|---|---|---|---|---|
| | | | | (Control | | |
| | Pain | | | Paw/ | | |
| | Model | | Acetic | Injured | | |
| | Tested | Oxaliplatin | Acid | Paw) | Carrageenan | TNBS |
| Signal/Method | White | P = 0.05 | N.S. | N.S. | P = 0.01 | P = 0.01 |
| Delivered | Noise | | | | | |
| | WAV 1 | N.S. | Not tested | Not tested | P = 0.05 | N.S. |
| | WAV 1′ | P = 0.01 | Not tested | Not tested | P = 0.01 | P = 0.01 |
| | WAV 4 | Not tested | N.S. | Not tested | N.S. | N.S. |
| | WAV 4′ | Not tested | N.S. | Not tested | P = 0.01 | N.S. |
| | WAV 2 | P = 0.05 | Not tested | N.S./N.S. | P = 0.01 | Not tested |
| | WAV 2′ | P = 0.01 | Not tested | P = 0.004/ | P = 0.01 | Not tested |
| | | | | N.S. | | |
| | WAV 5 | Not tested | N.S. | Not tested | Not tested | Not tested |
| | WAV 5′ | Not tested | N.S. | Not tested | Not tested | Not tested |
| | WAV 3 | P = 0.05 | Not tested | Not tested | Not tested | Not tested |
| | WAV 3′ | P = 0.01 | Not tested | Not tested | Not tested | Not tested |
| | WAV 6 | Not tested | N.S. | Not tested | Not tested | Not tested |
| | WAV 6′ | Not tested | N.S. | Not tested | Not tested | Not tested |

| | | | | | |
|---|---|---|---|---|---|
| ANOVA | Yes | Yes | Yes | Yes | Yes |
| Statistical Test | Dunnett's | Dunnett's | Students | Dunnett's | Dunnett's |
| | | | T-Test | | |
| Comparison Type | Multiple | Multiple | Pair-Wise | Multiple | Multiple |

In some embodiments, the signals have been modified to contain frequencies between DC and 6 kHz. Such modified signals may have the ability to reduce pain sensation and inflammation was measurably increased.

As shown in Table 1, in four out of five pain models used to assess pain reduction the modified signals (WAV 1′-WAV 6′) showed a consistent ability to reach a statistically significant value of activity, when compared to the full-frequency, unmodified signal (WAV 1-WAV 6). More specifically, in oxaliplatin (FIG. 19), carrageenan (FIGS. 20A-20B), and TNBS (FIG. 21) assays and the surgical neuropathy (FIGS. 22A-22B) assays, the modified signals achieved statistical significance at lower P values and had higher average values than the full-length, unmodified signals. The exception to this trend occurred in the TNBS model, in which both WAV 4 and WAV 4′ did not reach a statistically significant cut-off value (P=0.05).

The surgical neuropathy model (FIG. 22A-22B) achieved statistical significance in the post-treatment control paw (P=0.004) for WAV 2′ and trended towards significance in the injured paw group (P=0.06). The modest increase in applied pressure after WAV 2′ exposure in the control paw group indicated that the signal had an analgesic effect in a non-injured limb but did not significantly reduce the pain associated with tactile allodynia in the injured paw. The White Noise signal did not demonstrate any analgesic effect in either paw.

However, not all pain models showed a significant effect, such as the acetic acid abdominal contraction model (FIG. 23). None of the Signals (WAV 4-WAV 6 and WAV 4′-WAV 6′) including the White Noise signal, demonstrated a statistically significant inhibition in the abdominal contraction assay.

Given that modified signals consistently produced statistically significant effects in four out of five pain models (with the exception of White Noise and WAV 4), these results suggest that some signals with frequencies of DC—6 kHz can provide an efficacious reduction in pain sensation in these short-term pain studies. WAV 4, with a frequency range of DC—410 kHz and WAV 4′ (DC-2.02 kHz) suggest that there is a lower bound for frequency efficacy.

The results of the present examples indicate that these signals have pain reducing effects in specific pain models in rats without any apparent safety concerns.

Furthermore, modification of the original signals (DC—22 kHz) by reducing the frequency range (DC—6 kHz) produced a consistent pain inhibitory effect, highlighting the potential for further signal enhancement after recording. The ability to induce analgesia and reduce nociception via the application of a magnetic field, without (potential) systemic side-effects, would be a significant improvement in pain management, diversion control and stock maintenance of pharmaceutical compounds, without the limitations of systemic dilution, delivery and metabolites.

### FURTHER EXAMPLES

The following examples are illustrative of several embodiments of the present technology.
1. A method for reducing pain in a subject comprising administering to a subject one or more ulRFE signals.
2. The method of example 1, wherein the one or more ulRFE signals are administered using the EMulate Voyager system.
3. The method of example 1 or example 2, further comprising administering to the subject an opioid, a muscle relaxer, a steroid or an NSAID.
4. The method of any one of examples 1 to 3, wherein the one or more ulRFE signals are in the range of DC to 6 kHz.
5. The method of any one of examples 1 to 4, wherein the one or more ulRFE signals are administered for about 24 hours.
6. The method of any one of examples 1 to 5, wherein the subject is experiencing one or more of visceral pain, neuropathic pain, and inflammatory pain.
7. The method of any one of examples 1 to 6, wherein the subject does not exhibit any adverse events after administration of the one or more ulRFE signals.
8. A method for providing an analgesic effect in a subject comprising administering to the subject one or more ulRFE signals.
9. The method of example 8, wherein the one or more ulRFE signals are administered using the EMulate Voyager system.
10. The method of example 8 or example 9, further comprising administering to the subject an opioid, a muscle relaxer, a steroid or an NSAID.
11. The method of any one of examples 8 to 10, wherein the one or more ulRFE signals are in the range of DC to 6 kHz.
12. The method of any one of examples 8 to 11, wherein the one or more ulRFE signals are administered for about 24 hours.
13. The method of any one of examples 8 to 12, wherein the subject is experiencing one or more of visceral pain, neuropathic pain, and inflammatory pain.
14. The method of any one of examples 8 to 13, wherein the subject does not exhibit any adverse events after administration of the ulRFE signal.
15. Use of a system to administer one or more ulRFE signals to a subject having pain, wherein the system is a EMulate Voyager system.
16. The use of example 15, wherein the subject is also being treated with an opioid, a muscle relaxer, a steroid, or an NSAID.
17. The use of example 15 or example 16, wherein the subject does not exhibit any serious adverse events after administration of the one or more ulRFE signals.
18. A device, a system, and/or a kit for performing the method of any one of examples 1 to 14 or the use of any one of examples 15 to 17.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, additional embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” The word “coupled,” as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or” in reference to a list of two or more items, that word covers all of the following interpretations of the word: any of the items in the list, all of the items in the list, and any combination of the items in the list.

As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

From the foregoing, it will be appreciated that specific embodiments of the present technology have been described herein for purposes of illustration, but that various modifications may be made without deviating from the scope of the present technology. Accordingly, the present technology is not limited except as by the appended claims.

All of the above patents and applications and other references, including any that may be listed in accompanying filing papers, are incorporated herein by reference. Aspects of the present technology can be modified, if necessary, to employ the systems, functions, and concepts of the various references described above to provide yet further embodiments of the present technology.

These and other changes can be made to the present technology in light of the above Detailed Description. While the above description details some embodiments of the present technology and describes the best mode contemplated, no matter how detailed the above appears in text, the present technology can be practiced in many ways. Details of the signal processing system may vary considerably in its implementation details, while still being encompassed by the present technology disclosed herein. As noted above, particular terminology used when describing some features or aspects of the present technology should not be taken to imply that the terminology is being redefined herein to be restricted to any specific characteristics, features, or aspects of the present technology with which that terminology is associated. In general, the terms used in the following claims should not be construed to limit the present technology to the specific embodiments disclosed in the specification, unless the above Detailed Description section explicitly defines such terms. Accordingly, the actual scope of the present technology encompasses not only the disclosed embodiments, but also all equivalent ways of practicing or implementing the present technology under the claims.

I/We claim:

1. A method for reducing pain in a subject comprising administering to a subject one or more ultra-low radio frequency energy (ulRFE) signals.

2. The method of claim 1, wherein the one or more ulRFE signals are administered using a system comprising a battery-operated controller, an electromagnetic coil, and a battery charger.

3. The method of claim 1, further comprising administering to the subject an opioid, a muscle relaxer, a steroid or an NSAID.

4. The method of claim 1, wherein the one or more ulRFE signals are in the range of DC to 6 kHz.

5. The method of claim 4, wherein the one or more ulRFE signals are administered for about 24 hours.

6. The method of claim 1, wherein the subject is experiencing one or more of visceral pain, neuropathic pain, and inflammatory pain.

7. The method of claim 1, wherein the subject does not exhibit any adverse events after administration of the one or more ulRFE signals.

8. A method for providing an analgesic effect in a subject comprising administering to the subject one or more ulRFE signals.

9. The method of claim 8, wherein the one or more ulRFE signals are administered using a system comprising a battery-operated controller, an electromagnetic coil, and a battery charger.

10. The method of claim 8, further comprising administering to the subject an opioid, a muscle relaxer, a steroid or an NSAID.

11. The method of claim 8, wherein the one or more ulRFE signals are in the range of DC to 6 kHz.

12. The method of claim 11, wherein the one or more ulRFE signals are administered for about 24 hours.

13. The method of claim 8, wherein the subject is experiencing one or more of visceral pain, neuropathic pain, and inflammatory pain.

14. The method of claim 8, wherein the subject does not exhibit any adverse events after administration of the ulRFE signal.

15. Use of a system to administer one or more ulRFE signals to a subject having pain, wherein the system comprises a battery-operated controller, an electromagnetic coil, and a battery charger.

16. The use of claim 15, wherein the subject is also being treated with an opioid, a muscle relaxer, a steroid, or an NSAID.

17. The use of claim 15, wherein the subject does not exhibit any serious adverse events after administration of the one or more ulRFE signals.
